(19) United States
(12) Patent Application Publication
(10) Pub. No.: US 2013/0274142 A1
(43) Pub. Date: Oct. 17, 2013

(54) Cell Co-Culture Systems and Uses Thereof

(71) Applicant: Dana-Farber Cancer Institute, Inc., Boston, MA (US)

(72) Inventors: Constantine S. Mitsiades, Boston, MA (US); Douglas W. McMillin, Cambridge, MA (US); Joseph M. Negri, Jamaica Plain, MA (US); Nicholas Mitsiades, Roxbury, MA (US); Kenneth C. Anderson, Wellesley, MA (US)

(21) Appl. No.: 13/799,615

(22) Filed: Mar. 13, 2013

Related U.S. Application Data
(62) Division of application No. 12/525,394, filed on May 20, 2010.
(60) Provisional application No. 60/899,069, filed on Feb. 1, 2007.

(51) Int. Cl. G01N 33/50

(52) U.S. Cl. CPC G01N 33/5008
USPC 506/10

(57) ABSTRACT

The invention provides a cell co-culture for the selective evaluation of the response of a cell of interest in the co-culture, and methods of using the co-culture. The cell co-culture and the methods are suitable for large-scale/high throughput screening for compounds useful for affecting at least one biological function or event of at least one cell type in the co-culture. The invention further provides kits for using the screening assays.

### RELATED APPLICATIONS

This application claims the benefit of priority to U.S. Provisional Application No. 60/899,069 filed on 1 Feb. 2007, which application is incorporated herein, in its entirety by this reference.

### BACKGROUND OF THE INVENTION

Cells represent the primary building blocks of higher biological systems, such as tissues, organs, as well as entire microcellular organisms. In higher organisms, e.g., mammals, cells often interact with one mother for such important biological functions as transmitting signals and building macrostructures, including tissues. Cell interaction may also profoundly influence various disease states, such as infectious, immune and autoimmune disorders, primary site or metastatic cancers, thus it is often of great importance to study any specific biological problem in its in vivo context, or at least in a system that somewhat mimics or approximates its in vivo context.

However, due to many technical and theoretical difficulties, doing so is not always possible or practical.

For example, in the field of new drug development, traditional studies tend to focus on the effect of a candidate compound on a specific cell type of interest, in isolation from the general biological context in which the cell functions. In other words, this type of study, for various reasons, intentionally or accidentally omits the microenvironment in which the cell operates, and thus it may not come as a surprise when one identifies a promising drug candidate in the initial in vitro study, only to find in later stage drug development that the candidate drug fails in clinical trial.

One case on point is drug development for cancer treatment. Historically, the early stages of anti-cancer drug development have involved high-throughput screening of large libraries of compounds for potential in vitro activity against tumor cell lines. In these screening modalities, tumor cells are studied in conventional in vitro systems, where tumor cells are cultured in isolation from any other cell types with which they might interact in the in vivo local microenvironment of the tumor. These conventional screening strategies have included, e.g., the NCI60 panel of 60 tumor cell lines, which has been the basis for the anti-cancer screening program of the Developmental Therapeutics Program of the National Cancer Institute (NCI). Overall, the NCI60 panel and other similar screening programs in both academia and industry have been useful in identifying candidate anti-cancer compounds, many (but not all) of which have translated in clinical applications for systemic chemotherapy of human malignancies.

Unfortunately, systemic chemotherapy using anti-cancer compounds for human neoplasia, which may have been identified using such methods, is generally not curative. In fact, a key challenge identified in the oncology field for several years now is the contrast between the remarkable in vitro anti-tumor activity exhibited in the past by many conventional and investigational anti-cancer agents, and their typically less impressive clinical activity of these agents when they were eventually tested in clinical trials.

This kind of problem is by no means a unique phenomenon of cancer drug development. Most (if not all) drugs do not affect a single cell type, instead, they act on many different types of living cells in an entire organism. Thus the ultimate efficacy of a drug not only depends on its effect on its target cell, but also the influence of the microenvironment on the target cell. Thus arguably, all drug development faces the same issue, maybe to different extents. This problem is particularly acute in modern day drug development, where years (if not decades) of research and tremendous amount of human and financial resources are typically devoted to the process. A recent study by DiMasi et al. (Journal of Health Economics 22: 151-185, 2003) shows that the estimated average out-of-pocket cost per new drug is about US$403 million (year 2000 dollars), or double that amount if out-of-pocket costs are capitalized to the point of marketing approval at a real discount rate of 11%.

There is an urgent need, thus, for a system that better reflects the in vivo microenvironment in which cell-cell interactions take place. There also exists a need for a pathophysiologically relevant system to study a host of biological problems in the context of cell-cell interaction, such as screening for lead anti-cancer compounds which not only show in vitro screening activity but also have higher probability of in vivo efficacy.

### SUMMARY OF THE INVENTION

The present invention is based, at least in part, on the discovery of a cell co-culture system comprising two or more cellular compartments wherein at least one cellular compartment comprises a compartment-specific marker suitable for high throughput detection, kits for use in connection with the co-culture system and methods for using the systems and kits.

Accordingly, this invention provides a cell co-culture system comprising (1) a first cellular compartment having a compartment-specific marker for a biological activity of interest wherein said compartment-specific marker is suitable for, but not limited to, high-throughput detection; (2) a second cellular compartment and, (3) a detector suitable for detecting the compartment-specific marker in high throughput format.

In one aspect, the first cellular compartment comprises a tumor cell. In one embodiment, the tumor cell is from a tumor cell line, tissue sample (e.g., primary tumors and metastatic tumors), non-solid tumor (e.g., adult or childhood Acute Lymphoblastic Leukemia (ALL), adult or childhood Acute Myeloid Leukemia (AML), Chronic Lymphocytic Leukemia (CLL), Chronic Myelogenous Leukemia (CML), Hairy Cell Leukemia, AIDS-Related Lymphoma, adult or childhood Hodgkin's Lymphoma, adult or childhood Hon-Hodgkin's Lymphoma, T-Cell Lymphoma, Cutaneous Lymphoma, myeloproliferative disorders (e.g., polycythemia vera, essential thrombocythemia, chronic idiopathic myelofibrosis), myelodysplastic syndromes (e.g. essential thrombocytemia, polycythemia vera), Histiocytosis, and plasma cell dyserasias including Multiple Myeloma (MM), myeloma cell, leukemia cell, solid tumor (e.g., sarcoma or carcinoma of the bone, cartilage, soft tissue, smooth or skeletal muscle, CNS (brain and spinal cord), Peripheral Nervous System (PNS), head and neck, esophagus, stomach, small or large intestine, colon, rectum, GI tract, skin, liver, pancreas, spleen, lung, heart, thyroid, endocrine or exocrine glands, kidney, adrenals, prostate, testis, breast, ovary, uterus, and cervix). In another embodiment, the first cellular compartment comprises human cells. In another embodiment, the first cellular comprises non-human mammalian cells. In another embodiment, the first cellular comprises non-mammalian cells.

In another aspect, the first cellular compartment comprises a non-malignant cell. In one embodiment, the non-malignant cell is a bacterium, a fungal cell, a parasitic cell, an immortalized cell, a non-malignant tumor cell, immune system cell, a virally infected cell. In yet another embodiment, the non-malignant cell is a cell involved in inflammation (e.g., a B-lymphocyte, a T-lymphocyte, a Natural Killer (NK) cell, a macrophage, a monocyte, a neutrophil, an eosinophil, a basophil, a mast cell, or a dendritic cell). In another embodiment, the non-malignant cell is a CD4⁺ T-lymphocyte. In yet a further embodiment, the first cellular compartment comprises human cells. In another embodiment, the first cellular comprises non-human mammalian cells. In another embodiment, the first cellular comprises non-mammalian cells.

In another aspect, the cells of the second cellular compartment are cells of the same cell type(s) as those that interact in vivo with the cells of the first cellular compartment.

In another aspects, the first cellular compartment comprises a tumor cell, and the second cellular compartment comprises cells present in the microenvironment of the tumor cell in vivo. In one embodiment, the tumor cell is from a primary tumor or a metastatic tumor. In another embodiment, the tumor cell is a myeloma cell or a leukemia cell, and the cellular compartment comprises bone marrow stromal cells, mesenchymal cells, fibroblast cells, bone cells, endothelial cells, immune cells, nerve cells, glial cells, stellate cells, epithelial cells, and/or liver cells, such as hepatocytes.

In another aspect, the compartment-specific marker is a heterologous marker.

In another aspect, the compartment-specific marker is an energy-emitting reporter. In one embodiment, the energy-emitting reporter is a fluorescent protein. In another embodiment, the detectable product is a positron emitter.

In another aspect, the compartment-specific marker is an enzyme that converts a substrate to a detectable product. In one embodiment, the enzyme is a luciferase. In another embodiment, the detectable product is fluorescent.

In another aspect, the first cellular compartment further comprises an additional compartment-specific marker.

In another aspect, the second cellular compartment comprises a marker different from the compartment-specific marker. In one embodiment, the compartment-specific marker and the different marker can be independently monitored.

In another aspect, the compartment-specific marker is encoded by a heterologous polynucleotide introduced into the first cellular compartment. In one embodiment, the heterologous polynucleotide is introduced into cells on a plasmid. In another embodiment, the heterologous polynucleotide is introduced into cells on a vital vector by infection. In yet another embodiment, the viral vector is a retroviral vector, adenoviral vector, adeno-associated viral vector, herpes-simplex viral vector, or a lentiviral vector. In still another embodiment, the heterologous polynucleotide is integrated into the genome of the first cell compartment.

In another aspect, the compartment-specific marker produces a quantifiable signal linearly proportional to the number of viable cells in the first cellular compartment.

In another aspect, the compartment-specific market produces a quantifiable signal independent of the presence or absence of said second cellular compartment, or independent of the ratio of the first cellular compartment to said second cellular compartment.

In another aspect, the compartment-specific marker is non-harmful to cells, and does not itself appreciably affect the biological activity of interest.

In another aspect, the biological activity of interest is cell viability, cell proliferation, cell migration, cell adhesion, temporal and/or spatial organization of cell morphology, or cell differentiation.

In another aspect, the biological activity of interest is transcriptional activity of a promoter region of a gene of interest.

The invention also provides a method for identifying a compound useful for modulating a cellular biological activity of interest in cells of the first cellular compartment, the method comprising: (1) contacting a cell co-culture system of the invention with a test compound and (2) detecting the signal generated by the compartment-specific marker from the cell co-culture system in the presence and absence of the test compound; wherein a statistically significant difference in the signal after contact with the test compound compared to the signal in the absence of the test compound is indicative that the test compound is capable of modulating the cellular biological activity of interest in cells of the first cellular compartment in the presence of cells in the second compartment.

In one embodiment, the test compound is a synthetic compound, a natural compound, or a mixture of multiple compounds from either class thereof. In another embodiment, the test compound is tested at two or more different concentrations. In yet another embodiment, the test compound is from a chemical library, a polypeptide library, an antibody library, a small molecule library, a polynucleotide library, or a mixture thereof. In still another embodiment, the signal is a fluorescent signal. In still another embodiment, the cellular biological activity of interest is cell viability, cell proliferation, cell migration, cell adhesion, temporal and/or spatial organization of cell morphology, or cell differentiation. In still another embodiment, the method further comprises determining the ability of the identified test compound to affect the activity of the compartment-specific marker, wherein an identified test compound not substantially modulating the activity of the compartment-specific marker is useful for affecting the cellular biological activity of interest.

The invention also provides a method for identifying a compound useful for modulating a cellular biological activity of interest in the first cellular compartment, the method comprising: (1) contacting a cell co-culture system of the invention with a test compound; (2) contacting, under substantially the same conditions, a second cell culture comprising the first cellular compartment but not the second cellular compartment with the test compound; and (3) detecting the signal generated by the compartment-specific marker from the cell co-culture system and the second cell culture; wherein a statistically significant decrease in the signal from the cell co-culture system compared to that of the second cell culture is indicative that the test compound is useful for modulating the cellular biological activity of interest in cells of the first cellular compartment.

In one embodiment, the test compound is a synthetic compound, a natural compound, or a mixture thereof. In another embodiment, the test compound is tested at two or more different concentrations. In yet another embodiment, the test compound is from a chemical library, a polypeptide library, an-antibody library, a small molecule library, a polynucleotide library, or a mixture of multiple compounds from any class thereof. In still another embodiment, the signal is a fluorescent signal. In still another embodiment, the cellular biological activity of interest is cell viability, cell proliferation, cell migration, cell adhesion, temporal and/or spatial organization of cell morphology, or cell differentiation. In still another embodiment, the method further comprises determining the ability of the identified test compound to affect the activity of the compartment-specific marker, wherein an identified test compound not substantially modulating the activity of the compartment-specific marker is useful for affecting the cellular biological activity of interest. In still another embodiment, the cell co-culture system and the second cell culture are contacted by the test compound at substantially the same time.

The invention also provides a method for identifying a treatment useful for modulating a cellular biological activity of interest, the method comprising: (1) subjecting a cell co-culture system of the invention to said treatment; (2) detecting the signal generated by the compartment-specific marker from the cell co-culture system in the presence and in the absence of the treatment; wherein a statistically significant change in the signal after the treatment compared to that without the treatment is indicative that the treatment is useful for modulating the cellular biological activity of interest in cells of the first cellular compartment. In one embodiment, the treatment is radiation, light, heat, photodynamic therapy, cellular vaccine therapy, and/or cellular immune therapy.

The invention also provides a kit comprising: (1) a vector encoding a compartment-specific marker for a biological activity of interest, wherein said compartment-specific marker is suitable for high-throughput detection; and, (2) a medium suitable for co-culturing two or more cell compartments. In one embodiment, the vector is a plasmid, a retroviral vector, a lentiviral vector, an adenoviral vector, an adeno-associated viral vector, or a herpes-simplex viral vector. In another embodiment, the kit further comprises cell isolation means and/or means for introducing the vector into cells. In yet another embodiment, the two or more cell compartments comprise a tumor cell compartment.

The invention also provides a kit comprising: (1) tumor cells; and (2) non-tumor cells that interact with the tumor cells in vivo. In one embodiment, the kit further comprises a vector encoding a compartment-specific marker for a biological activity of interest, wherein the compartment-specific marker is suitable for high-throughput detection. In another embodiment, the vector is a plasmid, a retroviral vector, a lentiviral vector, an adenoviral vector, an adeno-associated viral vector, or a herpes-simplex viral vector. In yet another embodiment, the non-tumor cells are present in the microenvironment in which the tumor cells grow in vivo. In still another embodiment, the tumor cells are from a primary tumor site or a metastatic tumor site.

The invention also provides a method of identifying a compound that overcomes accessory cell-mediated tumor cell resistance to an anti-tumor compound, the method comprising: (1) contacting a cell co-culture system of the invention with a test compound and the anti-tumor compound, wherein said first cellular compartment comprises a tumor cell, and said second cellular compartment comprises non-tumor accessory cells, and, wherein the accessory cells confer accessory cell-mediated tumor cell resistance to the anti-tumor compound; and (2) detecting the signal generated by the compartment-specific marker from the cell co-culture system in the presence and absence of the test compound; wherein a statistically significant change in the signal after contacting with the test compound compared to that before contacting the candidate compound is indicative that the candidate compound overcomes accessory cell-mediated tumor cell resistance to the anti-tumor drug. In one embodiment, the method farther comprises the verification that the test compound does not substantially affect the signal generated by the compartment-specific marker in a manner disassociated from the biological endpoint that the marker is intended to measure from the cell co-culture system (e.g., in the absence of the anti-tumor drug).

The invention also provides a mammalian cell co-culture system comprising: (1) a tumor cell compartment having a compartment-specific bioluminescent marker; (2) a non-malignant accessory cell compartment without the compartment-specific bioluminescent marker; and, (3) a detector suitable for detecting the compartment-specific bioluminescent marker in a manner suitable in high throughput format. In one embodiment, the non-malignant accessory cell, compartment comprises one or more cells selected from the group consisting of: bone marrow stromal cells, mesenchymal cells, fibroblasts, adipocytes, bone cells, endothelial cells, pericytes, immune cells, liver cells, kidney cells, prostate cells, ovarian cells, cervical cells, cells of the central nervous system including brain and spinal cord neurons, muscle cells, stomach cells, esophageal cells, cells that interact with the tumor cell in vivo, and cells that may directly or indirectly affect cancer cell behavior. In another embodiment, the tumor cell compartment comprises a myeloma cell or a leukemia cell.

The invention also provides a method for identifying a compound useful for treating cancer, the method comprising: (1) contacting a mammalian cell co-culture system of the invention with one or more candidate compounds; (2) detecting the signal generated by the compartment-specific bioluminescent marker from the cell co-culture system in the presence and absence of the candidate compounds; wherein a statistically significant decrease in the signal after contacting with the candidate compound compared to that before contacting with the candidate compound is indicative that the candidate compound is useful for treating cancer. In one embodiment, the bioluminescent marker is a luciferase marker. In another embodiment, the bioluminescent marker is a luciferase-GFP marker. In another embodiment, the bioluminescent marker is a luciferase-neo marker. In yet another embodiment, the signal generated by the compartment-specific bioluminescent marker is detected by a bioluminescence-detecting device, a luminometer or a fluorometer.

The invention also provides a method for identifying a compound useful for treating cancer, the method comprising: (1) providing a cell co-culture system of the invention, and in parallel, a second cell culture comprising the tumor cell compartment but not the accessory cell compartment; (2) contacting, under substantially the same conditions, the cell co-culture system and the second cell culture with a candidate compound; (3) detecting the signal generated by the compartment-specific bioluminescent marker from the cell co-culture system and the second cell culture; wherein a statistically significant decrease in the signal from the cell co-culture system compared to that of the second cell culture is indicative that the candidate compound is useful for treating cancer. In one embodiment, the bioluminescent marker is a luciferase marker. In another embodiment, the bioluminescent marker is a luciferase-GFP marker. In another embodiment, the bioluminescent marker is a luciferase-neo marker. In yet another embodiment, the signal generated by the compartment-specific bioluminescent marker is detected by a bioluminescence-detecting device, a luminometer or a fluorometer.

### BRIEF DESCRIPTION OF THE DRAWINGS

FIGS. 1A-1D show a linear relationship between bioluminescence signal and cell number. In FIG. 1A, multiple myeloma (MM) cells expressing luciferase (MM-1S-GFP-luc) were plated in duplicate in a 96-well optical plate and assayed for bioluminescence signal at increasing cell numbers (1,500-100,000) and increasing luciferin substrate volume (0.2-10 μL per well of 7.5 mg/mL). Signal was measured using a bioluminescence-detecting device (Ivis® Imaging System), and the best fit line displayed for each condition (R²>0.94 for each line) is shown in FIG. 1B. In FIG. 1C, an equivalent cell number (1,500-100,000 cells per well) and luciferin substrate volumes (0.2-10 μL per well) were added to an optical plate and measured using a plate reader luminometer (R²>0.99 for each line). In addition, the bioluminescence signal was measured for MM-1S-GFP-luc cells in the presence and absence of HS-5 hone marrow stromal cells or “BMSCs” (10,000 cells per well) with increasing numbers of myeloma cells (1,500-100,000 cells per well). Linearity in bioluminescence signal was observed across all cell concentrations tested independent of stromal co-culture (FIG. 1D).

FIGS. 2A-2C show a comparison of cell viability as detected using MTT (3-(4,5-Dimethylthiazol-2yl)-2,5-diphenyltetrazolium bromide, a tetrazole) assay versus bioluminescence assay. MM-1S-GFP-luc cells were plated and treated with increasing concentrations of Dexamethasone (72 hrs exposure; FIG. 2A), Doxorubicin (48 hr exposure; FIG. 2B) or PS-341 (24 hr exposure; FIG. 2C) in both a standard MTT assay and in the bioluminescence assay (luminometer) in the absence of BMSCs. Survival of myeloma cells was compared between each assay for various anti-myeloma agents.

FIGS. 3A-3C show flow cytometry based evaluation of cell survival of GFP⁺ myeloma cells in the presence or absence of BMSCs. MM-1S-GFP-luc cells were plated in the presence or absence of GFP stromal cells, and treated with either Doxo or vehicle. Myeloma cells in co-culture could be distinguished from stromal cells by their GFP positivity (FIG. 3A). Viability of myeloma cells was quantified by the percent of Apo2.7 cells within the GFP⁺ cell compartment. The percentage of Apo2.7⁺ GFP⁻ myeloma cells plated in the absence of stroma (FIG. 3B) was compared to GFP⁺ myeloma cells plated in co-culture with HB-5 BMSCs (FIG. 3C). These results, obtained with a low throughput flow cytometry technique, are compared to those obtained with the high-throughput compartment specific technique.

FIGS. 4A-4B show the effect of the BMSC co-culture on myeloma cell proliferation. MM-1S-GFP-luc and the Dex-resistant version MM-1R-GFP-luc myeloma cells were plated in the presence and absence of HS-5 BMSCs (10,000 stromal cells per well) at increasing numbers of myeloma cells (1,500-20,000 cells per well) and incubated for 48 hrs. The number of MM-1S-GFP-luc (FIG. 4A) and MM-1R-GFP-luc cells (FIG. 4B) were assayed by luminometer bioluminescence. The bioluminescence signal was greater in all co-culture-conditions for both MM-1S and MM-1R cells compared to cells cultured without BMSCs, indicating both cell lines are responsive to BMSC stimulation.

FIGS. 5A-5F show that BMSCs confer protection against specific anti-myeloma agents. MM-1S-GFP-luc (FIGS. 5A, 5C, and 5E) and MM1R-GFP-luc cells (FIGS. 5B, 5D, and 5F) were plated in the presence or absence of HS-5 BMSCs (10,000 per well) and treated with Dex (FIGS. 5A and 5B), Doxo (FIGS. 5C and 5D) or PS-341 (FIGS. 5E and 5F). BMSCs confer protection to MM-1S myeloma cells in response to treatment with Dex (FIG. 5A) and Doxo (FIG. 5C) but not the proteasome inhibitor PS-341 (FIG. 5B). BMSCs have little effect on the Dex-resistant cell line, MM-1R, in response to Dex treatment (FIG. 5B), but cooler protection against Doxo (FIG. 5D). In addition, BMSCs confer no protection to treatment with PS-341 in MM-1R cells (FIG. 5F). The results obtained in FIG. 5C and FIG. 5D with a high-throughput compartment specific technique are comparable to those obtained with the lower-throughput flow cytometry technique (FIG. 3).

FIGS. 6A-6D show protection of myeloma cells from Doxo treatment independent of the stromal cell line type they are co-cultured with MM-1S-GFP-luc cells were plated in the presence or absence of BMSCs (10,000 stromal cells per well) and treated with increasing doses of Doxo. KM101(FIG. 6A), KM103 (FIG. 6B), KM104 (FIG. 6C) and KM105 (FIG. 6D) BMSC lines were assayed for the magnitude of protection against Doxo treatment. All BMSC lines conferred protection against Doxo compared to myeloma cells treated in the absence of BMSCs.

FIGS. 7A and 7B show the effect of BMSCs on leukemia cell proliferation. Luciferase positive KU812F and K562 leukemia cell lines were plated in the presence and absence of HS-5 BMSCs (10,000 stromal cells per well) at increasing numbers of leukemia cells (1,500-20/100 cells per well) and incubated for 48 hrs. The number of KU812F-luc (FIG. 7A) and K562-luc cells (FIG. 7B) were assayed by luminometer bioluminescence. K812F-luc cell responsiveness was greater at lower cell numbers and less viable at higher cell numbers, where as K562-luc cells remained unresponsive at all cell concentrations plated.

FIGS. 8A-8F show that BMSCs confer protection to leukemia cell lines against various anti-leukemia agents. KU812F-luc (FIGS. 8A, 8C, and 8E) and K562-luc cells (FIGS. 8B, 8D, and 8F) were plated in the presence or absence of HS-5 BMSCs (10,000 per well) and treated with Ara-C (FIGS. 8A and 8B), Imatinib (FIGS. 8C and 8D) or Doxo (FIGS. 8E and 8F). BMSCs confer protection to KU812F cells in response to treatment with AraC (FIG. 8A) and Imatinib (FIG. 8C) but not Doxo (FIG. 8E). For K562 cells, BMSCs confer protection against Ara-C (FIG. 8B), but do not confer protection against Imatinib (FIG. 8D) or Doxo (FIG. 8F).

FIGS. 9A and 9B show the effect of blocking IL-6 or IL-6R using their respective blocking antibodies on the HS-5 stromal cell-mediated tumor resistance to drug (Doxo) treatment.

FIG. 10 describes an example of compartment-specific bioluminescence (CS-BLI). Marked tumor cells emit bioluminescence signal proportional to the number of viable cells after the addition of substrate. Unmarked stromal cells alone do not emit any bioluminescence signal when substrate is added. Marked tumor cells mixed with stromal cells results in a bioluminescence signal proportional to only the viable tumor cells in culture. Using this application, the interaction of tumors with the bone marrow microenvironment can be assessed in the setting of stromal protection of tumors to various anti-cancer agents.

FIG. 11 shows the results of viability measurement using Cell Titer Glo (CTG) compared to viability measurement using the addition of luciferin for detecting viable cells in the CS-BLI platform (E). Signal was normalized to the highest value of each curve.

FIGS. 12A-12F show the effect of BMSCs on myeloma cell proliferation/viability. Luciferase positive MM.1S, MM.1R, KMS-18, OPM2, KU812F, and K562 cell lines were plated in the presence and absence of HS-5 BMSCs (10,000 stromal cells per well) at increasing numbers of myeloma cells (1500-20,000 cells per well) and incubated for 48 hrs. The number of MM.1S-GFP-luc (A) MM.1R-GFP-luc (B) KMS18-GFP-luc (C) OPM2-GFP-luc (D) KU812F-luc-neo (E) and K562-luc-neo cells (F) were assayed by compartment-specific bioluminescence measurement using plate reader luminometer. MM.1S, MM.1R, and KMS18 cells responded to the presence of stromal cells resulting in increased viability signal following 48 hrs of coculture at all tumor cell concentrations tested. In addition, OPM2. KU8122F and K562 cells remained unresponsive at low cell concentrations and had lower viability for higher cell concentrations.

FIGS. 13A-13C show a comparison of drug sensitivity of GFP-luc cells using standard assays. MM.1S cells stably expressing a GFP-luciferase fusion construct and their parental untransfected MM.1S cells were treated with Doxo (48 hrs; panel A) and PS-341 (24 hrs; panel B) and the viability assessed for both cell lines by MTT assay. The parental cell line and the GFP-luc expressing cell line responded similarly to both Doxo and PS-341, indicating that the GFP-luc expression construct has little affect on their drug responsiveness. In addition, MM1S-GFP-luc cells were plated and treated with increasing concentrations of the proteasome inhibitor bortezomib (PS-341) (24 hr; panel C) and there response was evaluated with both a CellTiterGlo assay and the CS-BLI technique in the absence of BMSCs.

FIGS. 14A-14C shows an analysis of sensitivity of MM1S-GFP-luc cells (primary cell compartment) to Dexamethasone, using the CS-BLI technique, in the presence and absence of stromal cells from various sources. The second cell compartment in the co-culture system was either the HS-5 stromal cell line (A) stromal cells from a normal donor (B), or stromal cells from MM patients (C).

FIGS. 15A-15C shows an analysis of sensitivity of MM1S-GFP-luc cells (primary cell compartment) to PS-341. using the CS-BLI technique, in the presence and absence of stromal cells from various sources. The second cell compartment in the co-culture system was either the HS-5 stromal cell line (A) stromal cells from a normal donor (B), or stromal cells from MM patients (C).

FIGS. 16A-16C shows an analysis of sensitivity of MM1S-GFP-luc cells (primary cell compartment) to Doxo, using the CS-BLI technique, in the presence and absence of stromal cells from various sources. The second cell compartment in the co-culture system was either the HS-5 stromal cell line (A) stromal cells from a normal donor (B), or stromal cells from MM patients (C).

FIGS. 17A-H show that BMSCs confer to leukemia cell lines protection against various anti-leukemia agents, KU812F-luc (A, C, E, G) and K562-luc cells (B, D, F, H) were plated in the presence or absence of HS-5 BMSCs (10,000 per well) and treated with Ara-C (A, B), Imatinib (C, B), Doxo (E, F) or nilotinib (G, H). BMSCs confer protection to KU812F cells in response to treatment with AraC (A), Imatinib (C) and nilotinib (H) but not Doxo (E). For K562 cells, BMSCs confer protection against Ara-C (B), but do not confer protection against Imatinib (D), Doxo (F) or nilotinib (H).

FIGS. 18A-F show that co-culture of solid tumors with BMSCs provides differential protection of solid tumor cell lines to AraC and Doxo, MDA-M8-231met-luc-neo (A, B), A375-luc-neo (C, D) and FRO-luc-neo (E, F) cells were plated either alone or mixed with HS-5 BMSCs (10,000/well) and treated the following day. The cultures of tumor cells and their co-cultures with BMSCs were incubated for an additional 48 hrs. BMSCs confer modest protection to MDA-MB-231-met cells in response to both AraC (A) and Doxo (B). BMSCs provided a considerable level of protection to A375 cells in responses to AraC (C) hot provided no protection against Doxo (D). In contrast, BMSCs confer no protection to FRO cells in response to AraC (E) but confer modest protection against Doxo (F).

FIGS. 19A-D show that a high-throughput screen of a library of kinase inhibitors can identify compounds that are active both is the presence or absence of BMSCs, compounds that are less active in the presence of BMSCs, and compounds that are more active in the presence of BMSCs. Luciferase positive MM.1S (A) MM.1R (B) and KU812F (C) cells were screened against a panel of kinase and phosphatase inhibitors in the presence and absence of stromal cells. Cells were cultured in the presence of drug for 48 hrs and the viability assessed using CS-BLI bioluminescence measurement. Survival of tumor cells were normalized to DMSO controls in the absence of stromal cells. This figure (and FIGS. 20-22) depicts representative results, for each cell line, with emphasis on examples of inhibitors which exhibited (in at least one concentration of treatment, either in the presence or absence of stromal cells) <50% reduction in tumor cell viability and/or significant difference in their response to a particular inhibitor in the presence vs. absence of stromal cells. Additional compound libraries were screened in a 384-well, high-throughput manner and the survival signal between MM.1S-GFP-luc cells in the presence and absence of HS-5 cells was quantified following exposed to each compound for 48 hrs (FIG. 19D).

FIG. 20 shows sensitivity of MM.1S cells to a set of representative kinase inhibitors in the presence and absence of stroma by measuring the average percent viability of MM.1-GFP-Luc cells to kinase inhibitors in the presence and absence of stroma cells.

FIG. 21 shows sensitivity of MM.1R cells to a set of representative kinase inhibitors in the presence and absence of stroma by measuring the average percentage viability of MM.1R-GFP-Luc cells to kinase inhibitors in the presence and absence of stroma cells.

FIG. 22 shows sensitivity of KU812F cells to a set of representative kinase inhibitors in the presence and absence of stroma by measuring the average percentage viability of KU812F-Luc-neo cells to kinase inhibitors in the presence and absence of stroma cells.

FIG. 23 shows a time-lapse CS-BLI application for measuring MM cell viability in response to PS-341 across several time points. The MM cell lines MM.1S-GFP-luc (A) and OPM-2-GFP-luc (B) were plated at 2,000 cells per well in a 96-well optical plate, treated with increasing doses of PS-341 and luciferin substrate added at time 0. Cell viability was assessed serially up to 24 hrs by measuring bioluminescence on a Luminoscan plate reader and signal normalized to non-drug treated controls.

FIG. 24 shows a time-lapse CS-BLI application for measuring MM cell viability in response to Doxorubicin across several time points. The MM cell lines MM.1S-GFP-luc (A) and OPM-2-GFP-luc (B) were plated at 2,000 cells per well in a 96-well optical plate, treated with increasing doses of Doxorubicin and luciferin substrate added at time 0. Cell viability was assessed serially up to 48 hrs by measuring bioluminescence on a Luminoscan plate reader and signal normalized to non-drug treated controls.

FIG. 25 shows a time-lapse CS-BLI application for measuring MM cell viability in response to PS-341 (A), Doxorubicin (B), and Dex (C) across several time points in the presence or absence of stromal cells. Cultures were treated with increasing doses of PS-341 (A) Doxorubicin (B) or Dexamethasone (C), detection substrate added at time 0, and cell viability assessed serially for up to 48 hrs by measuring bioluminescence on a Luminoscan plate reader and signal normalized to non-drug treated controls in the absence of stromal cells.

FIG. 26 shows compartment-specific bioluminescence imaging application for quantification of tumor cell viability in co-cultures with immune cells. Tumor cells were plated at the cell number indicated in the presence and absence of 10000 peripheral blood mononuclear cells (PBMCs). Luciferin was immediately added, cultures incubated for 30 min at 37° C. and samples read on a luminometer. Bioluminescence signal for MM.1S-GFP-luc (A) and KU812F-luc-neo (B) cells stably expressing luciferase remained linear across a range of cell numbers and was equal both in the presence or absence of PBMCs. Each condition was run in triplicate.

FIG. 27 shows the application of CS-BLI for evaluation of specific killing of tumor cells by immune effector cells. PBMCs were isolated from a healthy donor, stimulated for 24 hrs with 10 ng/mL of IL-2 and then combined in culture with 5000 tumor target cells. The human multiple myeloma line, MM.1S-GFP-luc (A), was used as target cells and combined with PBMCs at 1:1, 1:5, 1:10, 1:20 and 1:40 ratios. The basophilic leukemia line, KU812F-luc-neo (B), was used as larger cells and combined with PBMCs from the same donor at 1:1, 1:5, 1:10, 1:20 and 1:40 ratios. Viability of tumor cells was assessed using bioluminescence detection after the addition of luciferin substrate following 4 hours of co-culture with immune effector cells.

FIG. 28 shows time-lapse CS-BLI analysis of the activity of PBMCs to kill myeloma and leukemia cells. PBMCs were isolated from a healthy donor, stimulated for 24 hrs with IL-2 and combined in culture with tumor target cells. The human multiple myeloma line, MM.1S-GFP-luc (A), was used as target cells and combined with PBMCs at 1:1, 1:5, 1:10, 1:20 and 1:40 ratios. The basophilic leukemia line, KU812F-luc-neo (B), was used as target cells and combined with PBMCs from the same donor at 1:1, 1:5, 1:10, 1:20 and 1:40 ratios. Viability of tumor cells was assessed serially at 1, 2, 3, and 4 hours of co-culture using CS-BLI following the addition of luciferin substrate at time 0.

FIG. 29 shows the effects of drug pretreatment of PBMCs for their anti-myeloma activity. PBMCs were isolated from a healthy donor, stimulated for 24 hrs with IL-2 in the presence of either 1 μM CC5013 (A), 1 μM CC4047 (B), 10 nM PS-341 (C), or 50 nM Dexamethasone (D). PBMCs were washed and combined in culture with MM.1S-GFP-luc target cells at the 1:1, 1:2.5, 1:5, 1:10. 1:20 and 1:40 target to effector ratios. PBMC pretreatment with CC5013 did not have a significant impact on either increasing or decreasing the activity of the PBMCs to kill tumor targets (A), PBMC pretreatment with CC4047 did not have a significant impact on increasing or decreasing the activity of the PBMCs to kill tumor targets (B), PBMC pretreatment with PS-341 did have a significant impact on blocking the activity of PBMCs to kill tumor targets (C) and PBMCs pretreated with Dex did not have a significant difference blocking or stimulating the activity of PBMCs to kill tumor targets (D).

FIG. 30 shows the effect of drug pretreatment on PBMCs for anti-leukemia activity. PBMCs were isolated from a healthy donor, stimulated for 24 hrs with IL-2 with either 1 μM CC5013 (A), 1 μMCC4047 (B), 10 nM PS-341 (C), or 50 nM Dexamethasone (D). PBMCs were washed and combined in culture with KU812PF-luc-neo target cells at the 1:1, 1:2.5, 1:5, 1:10, 1:20 and 1:40 target to effector ratios. PBMC pretreatment with CC5013 did not have a significant impact increasing or decreasing the activity of the PBMCs to kill tumor targets (A), PBMC pretreatment with CC4047 did have a significant impact on increasing the activity of the PBMCs to kill tumor targets (B), PBMCs pretreatment with PS-341 did have a significant impact on blocking the activity of PBMCs to kill tumor targets (C) and PBMC pretreatment with Dexamethasone did not have a significant impact on blocking or stimulating the activity of PBMCs to kill tumor targets (D).

FIG. 31 shows how drug pretreatment of PBMCs alone vs. myeloma cells alone vs. both affects the anti-myeloma killing activity of PBMCs. PBMCs were isolated from a healthy donor, stimulated tor 24 hrs with IL-2 in the presence or absence of 1 μM CC4047. MM.1S-GFP-luc cells were cultured in the presence or absence of 1 μM CC4047. PBMCs and MM.1S-GFP-luc cells were washed to remove drug and combined in culture with target MM.1S-GFP-luc target cells, either pretreated with 1 μM CC4047 or without, and combined At 1:1, 1:2.5, 1:5, 1:10, 1:20 and 1:40 target to effector ratios in the absence of drug. PBMCs pretreated with CC4047 have no significant increasing in activity of the PBMCs to kill tumor targets, but MM.1S-GFP-luc cells pretreated had a significant increase in the ability to kill tumor targets.

FIG. 32 shows how the anti-myeloma cytotoxic activity of PBMCs is influenced by concurrent exposure of the co-culture with various drug treatments. PBMCs were isolated from a healthy donor, stimulated for 24 hrs with IL-2. PBMCs were combined in culture with MM.1S-GFP-luc target cells at the 1:1, 1:2.5, 1:5, 1:10, 1:20 and 1:40 target to effector ratios in the presence or absence of 2 μM CC5013 (A), 2 μM CC4047 (B), 20 nM PS-341 (C), or 100 nM Dex (D) during co-culture. Cultures treated with CC5013 had an increase in the activity of the PBMCs to kill tumor targets (A), cultures treated with CC4047 also had increased activity of the PBMCs to kill tumor targets (B), cultures treated with PS-341 had decreased activity of PBMCs to kill tumor targets (C) and cultures treated with Dexamethasone had no significant difference blocking the activity of PBMCs to kill tumor targets (D).

FIG. 33 shows how the anti-leukemia cytotoxic activity of PBMCs is influenced by concurrent exposure of the co-culture with various drug treatments. PBMCs were isolated from a healthy donor, stimulated for 24 hrs with IL-2. PBMGs were then washed and combined in culture with KU812F-luc-neo target cells at the 1:1, 1:2.5, 1:5, 1:10, 1:20 and 1:40 target to effector ratios in the presence or absence 2 μM CC5013 (A), 2 μM CC4047 (B), 20 nM PS-341 (C), or 100 nM Dex (D). Cultures treated with CC5013 had a significant difference increasing the activity of the PBMCs to kill tumor targets (A), cultures treated with CC4047 also had a significant difference increasing the activity of the PBMCs to kill tumor targets (B), cultures treated with PS-341 had a significant difference blocking the activity of PBMCs to kill tumor targets (C) and cultures treated with Dex had no significant difference blocking the activity of PBMCs to kill tumor targets (D).

FIG. 34 shows the effect of stromal cells on the cytotoxic activity of immune cells against myeloma and leukemia targets. HS-5 stroma cells were plated in a 384-well plate and cultured overnight. MM.1S-GFP-luc (A) or KU812F-luc-neo cells (B) were combined with PBMCs at 1:1, 1:25, 1:5, 1:10, 1:20 and 1:40 ratios in the presence or absence of stromal cells and/or 2 μM CC4047. CS-BLI was used to evaluate the ability of stoma cells to affect the killing of MM.1S-GFP-luc (A) or KU812F-luc--neo (B) by PBMCs.

FIG. 35 shows results of CS-BLI measurement of anti-tumor activity of immune cells following depletion and selection of specific lymphocyte subsets. Normal donor PBMCs were isolated using Ficoll gradient separation and specific lymphocyte subsets depleted (A) or selected(B) using Miltenyi microbeads for CD4, CD8, and CD56, CD4+/−, CD8+/−, and CD56+/− PBMC subsets were then cultured overnight in the presence of IL-2. The following day MM.1S-GFP-luc targets were plated at various target: effector ratios in the presence of IL-2 with the depleted (A) or selected (B) PBMC subsets. Viable MM.1S-GFP-luc cells were measured at 6 hrs.

### DETAILED DESCRIPTION OF THE INVENTION

The invention provides a cell co-culture system suitable for the rapid and sensitive detection and/or quantification in a high throughput format of a cellular activity of interest in one or more cell-types of interest in the co-culture. According to the invention, the co-culture system comprises two or more cell populations (compartments), wherein at least one of the populations comprises a compartment-specific marker suitable for high throughput applications, for detecting a cellular activity of interest in that cellular compartment. The compartment-specific marker allows the cells of interest to be detected separately from the accessory cells, yet at the same time, allows the biological activity of interest to be studied in the context of the accessory cells.

The co-culture system further comprises a means for rapidly detecting the compartment-specific marker, and optionally for quantifying the signal. The invention further provides various methods, including high throughput methods, of using the co-culture system of the invention, and kits for using the cell co-culture system and methods of the invention.

The systems, kits and methods of the invention provide a patho-physiologically relevant model for studying the effect of a co-cultured cell type, referred to herein as an accessory cell type or accessory compartment, on the activity of a cell type of interest, including the response of a cell type of interest to test compounds, changes in co-culture conditions, such as additions of cytokines, growth factors, differentiation factors, nutrients, concentrations of oxygen, exposure to visible, infra-red or ultra-violet frequencies, irradiation and any other stimuli that can modify cell behavior, and the like. For example, as described in detail herein, the systems and methods of the invention are useful to study the effect of a treatment, including pharmacological and non-pharmacological treatments, on a cellular compartment of interest in the presence of one or more accessory cell populations, including populations that occur in die milieu of the cell of interest in vivo. In particular, the systems, kits and methods of the invention are useful to screen for and identify therapeutic compounds, including anti-neoplastic molecules in a pathophysiologically relevant model. The requirement that the compartment-specific marker be suitable for high throughput applications permits the use of the systems, kits and methods for the rapid analysis of large numbers of samples.

In its simplest form, the co-culture system is a dual compartment system comprising a cell type of interest, also referred to as a compartment of interest or cellular compartment of interest that is stably transfected with a compartment-specific marker for the biological activity of interest, and a cell type, referred to as an accessory cell, or whose effect on the cell type of interest it is desired to study, which may or may not comprise a different compartment-specific marker. The compartment-specific marker in the cellular compartment of interest must be one that is amenable to high throughput applications.

According to the invention, the co-culture system may be expanded to include two or more compartments of interest, two or more accessory compartments, or both. Where the system comprises multiple compartments of interest, each compartment that is desired to be studied comprises at least one compartment-specific marker for the activity of interest.

In certain embodiments, one or more cell compartments of interest may be present in the cell co-culture system, each with at least one different/distinct marker, but may share an identical marker. In the latter case, the shared marker would be useful to monitor all cell compartments of interest together, while their distinctive compartment-specific markers may be used to trace each compartment of interest separately. One exemplary system of multiple cell compartments that are of interest includes immune therapy against cancer cells or virally-infected cells (such as HIV-infected cells). In cancer immunotherapy, one or more tumor antigens or epitopes may be present on tumor surface, yet none of which may be immunogenic enough to trigger a recognition of such tumor antigens/epitopes by the antigen presenting cells (APCs), and thus no host immune cells (such as T-cells and/or B-cells) are activated. Such immune tolerance may be broken by administering one or more compounds, such as antibodies against the tumor antigens. Thus in this system, it would be advantageous to monitor both the activity and/or proliferation of the T- and/or B-cells (through, for example, a T- and/or B-cell compartment-specific marker), and the survival of tumor cells (through, for example, a minor cell compartment-specific marker), in the presence of APCs (the accessory cell compartment). Although the same experiment may be approximated by two separate cell co-cultures, e.g., one by a co-culture of tumor cells with APC cells (such as immature dendritic cells), and the other by a co-culture of the activated APC cells (such as mature dendritic cells) and immature T- and/or B-cells, the overall effect of the test compound (such as the anti-tumor antibody) may best be studied in a 3- or 4-compartment co-culture system of the invention.

Further, the co-culture systems of the invention can utilize two-dimensional or three-dimensional co-culture. Exemplary three-dimensional co-cultures include cell co-cultures used in colony formation assays. Another embodiment includes a lattice of extra-cellular matrix proteins used for culturing cells in three-dimensional space.

Any cell type that can be stably transfected with a suitable compartment-specific marker may be used in the co-culture system of the invention. Any cell type whose effect on the cells of interest it is desired to study can be utilized in the accessory cell compartment. In co-cultures in which it is desired to study viral infection, any host cell of interest can be utilized, as long as it is capable of being infected by the virus of interest; and any cell whose effect on the ability of the virus to infect the host cell type of interest is desired to be studied can be utilized as the accessory cell type. In a viral infection model, i.e., when the virus is added to a co-culture comprising the viral host cell, the virus comprises a compartment-specific marker so that infected host cells can be distinguished from uninfected host cells. The compartment-specific marker may be a gene in its natural version, or engineered in a modified version.

The co-culture system of the invention may be prokaryotic or eukaryotic. Prokaryotic cells useful in the systems, kits and methods of the invention include pathogens such as bacteria.

Eukaryotic cells that are useful in the invention can be from any species. Simple eukaryotic cells useful in the systems, kits and methods of the invention include parasites or fungi. Particularly useful eukaryotic cells are from mammals. Mammalian cells that are useful in the invention include but are not limited to mouse, rat, hamster, rabbit, dog, cat, pig, goat, cow, non-human primates (e.g., monkey, ape, gorilla, etc.), or, preferably, humans. The cells of interest can be from the same species or a different species than the accessory cells. For example, the cells of interest and the accessory cells both may be human cells. Alternatively, the cells of interest may be human cells and the accessory cells may be mouse cells. Where there are multiple compartments of interest, the compartments may be from the same species or from different species. Likewise, where there are multiple accessory compartments, they may be from the same species or from different species.

Cells that are useful in the co-culture system of the invention may be undifferentiated, (e.g., stem cells), partially differentiated (e.g., progenitor cells) or fully differentiated. They may be from cultured cell lines or from primary tissue samples, especially samples from humans. The cells may be transformed or immortalized or untransformed.

The co-culture system of the invention may be used with a wide range of sizes of cell populations. The ability to use a low cell number is advantageous, particularly for the use of cells from primary tissue samples where the number of cells available for testing is limited. Those of skill in the art will appreciate that when using a small number of cells it is necessary that a compartment-specific marker be selected which is detectable under conditions of low cell number. The upper limit of the number of cells in the co-culture is affected by parameters such as the well size itself as well as the proliferation rate of the cells, duration of the experiment, etc. The systems and methods of the invention, thus, are useful for experiments in which the size of one or more compartments in the co-culture is varied.

Cells that are useful in the systems and methods of the invention also include cells comprising genetic manipulations in addition to comprising the one or more compartment-specific markers. According to the invention, cells in a compartment of interest or in an accessory compartment may be genetically modified to express a heterologous gene product, to overexpress a gene product from an endogenous gene or from a heterologous gene, or to reduce or prevent the expression of a gene. Such expression, over-expression or reduced expression can be constitutive or inducible. Inducible expression of compartment-specific marker(s) can be used to probe the activity of specific molecular pathways. Inducible expression systems are well-known to those of skill in the art.

Cells for use in the co-culture system of this invention can be genetically modified using any of many means for genetic modification of a cell known in the art. The gene product to be expressed, over expressed or reduced may be nucleic acid or protein. In the case of a nucleic acid, the gene product can be DNA, including cDNA, or RNA, including messenger RNA (mRNA), transfer RNA (tRNA), ribosomal RNA (rRNA), microRNA, short interfering RNA (siRNA), shRNA (short hairpin RNA). For example, techniques for expressing or over-expressing a gene product include transfection, transformation, or infection with appropriate vectors. Techniques for reducing or preventing the expression of a gene product include but are not limited to antisense, including single-stranded or double-stranded antisense molecules, antisense oligonucleotides having modified backbones or nucleobases including 2′ modifications such as 2′MOE, locked nucleic acids (LNA) and the like, siRNA, microRNA, or gene knock-out. Those of skill in the art will recognize other techniques that are useful for genetic manipulation of the cells in the co-culture.

In certain embodiments, the co-cultured accessory cells may be manipulated to examine the effect of such manipulation on the cell type(s) of interest. Thus the co-cultured accessory cells may contain any of the art-recognized conditionally inducible promoters (such as heat, shock promoters, TetON/OFF promoters, lac promoters, FLF/FRT flanked promoters, etc.) that can be turned on or off in an inducible and/or reversible fashion. Modification of the accessory cells to over-express genes or knock down gene expression through, for example, siRNA methods could also shed light on the mechanisms of cell-cell interactions between the cell type(s) of interest and the accessory cell.

There is at least one very clear distinction between the current invention and any prior co-culture assays for quantification of cell viability: the latter do not distinguish between the types of cells in the co-culture. In contrast, the systems and methods of the invention are clearly designed to allow for selective-evaluation of the behavior of one (or more) cell type compartment of interest (for instance, a population of tumor cells) as they interact with another cell compartment (e.g., a population of stromal cells or, more generally, a population of non-malignant accessory cells), through the use of compartment-specific markers.

Thus the systems and methods of the invention comprise a cellular compartment of interest wherein the cells in the compartment are stably transfected with a compartment-specific marker that is amenable to detection in high throughput. The marker allows specific detection of the cell type of interest within a mixed co-culture.

As used herein, “high throughput screening” (HTS) refers to a process that uses a combination of modern robotics, data processing and control software, liquid handling devices, and/or sensitive detectors, to efficiently process a large amount of (e.g., thousands, hundreds of thousands, or millions of) samples in biochemical, genetic or pharmacological experiments, either in parallel or in sequence, within a reasonable short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μL, 200 μL, 100 μL, 50 μL or less. Through this process one can rapidly identify active compounds, small molecules, antibodies, proteins or polynucleotides which modulate a particular biomolecular/genetic pathway. The results of these experiments provide starting points for further drug design and for understanding the interaction or role of a particular biochemical process in biology. Thus “high throughput screening” as used herein does not include handling large quantities of radioactive materials, slow and complicated operator-dependent screening steps, and/or prohibitively expensive reagent costs, etc.

Any compartment-specific marker amenable to high throughput detection may be used with the systems, kits, and method of the instant invention. Markers that require cell lysis, that may freely diffuse from the accessory cells of the co-culture into the medium creating a high background signal (such as those used in the MTT assay or the Alamar Blue assay) or that can be taken up by other cell compartments, particularly the accessory compartment (such, as H3) are not compartment-specific and, thus, not suitable for use in the systems, kits and methods of the invention. Similarly, markers that require complex or time-consuming procedures (such as cell surface stains including antibodies and other binding reagents for, e.g., flow cytometry), that would be unsafe in large quantities (such as radioactive markers) or that require a significant amount of operator-dependent manipulation, which also is disadvantageous because it can produce variable results depending on operator experience) also are not suitable for use in the systems, kits and methods of the invention.

Compartment-specific markers of the invention may be heterologous, i.e., a marker that is introduced into the cells of the compartment of interest, whether or not the marker occurs endogenously in the cell, or endogenous. The important consideration is that the market is compartment-specific, and changes in the level of the signal from the marker accurately reflect changes in the biological activity of interest.

For example, certain cell types, such as certain tumor cells, may express or overexpress an endogenous protein not detestably expressed in normal cells (e.g., tumor markers or tumor antigens).

As will be appreciated by those of skill in the art, the choice of marker may also depend on the specific biological activity of interest. When it is desirable to detect cell viability (as in methods identifying cytotoxic compounds) the marker should be a biological marker that is detectable only in a living cell and not in dead or unmarked cells. Further, for the marker to accurately reflect changes in the number of viable cells, particularly where it is desired to quantify the number of cells, the compartment-specific marker whose expression level in viable cells is stable, i.e., whose expression level does not fluctuate in response to cellular or experimental conditions other than viability. For example, markers whose expression level fluctuates during different stages of the cell cycle, cell maturation or cell differentiation would not be suitable for such viability assay.

In certain embodiments, the compartment-specific marker, when stably integrated into the cells, allows the detection of a signal by the detector from about 100 cells with the stably integrated marker, or about 500 cells, 1,000 cells, 2,000 cells, 4,000 cells, 8,000 cells, 15,000 cells, 30,000 cells, 50,000 cells, 100,000 cells, or 200,000 cells with the marker.

Useful markers where cell viability is of interest include but are not limited to certain energy-emitting reporter proteins, or certain enzymes, including enzymes in bioluminescent systems, that function in a living cell. Alternatively, a marker that is only functional outside the cell may be used to monitor the amount of dead or damaged cells (such as when the marker is released upon cell lysis, and becomes functional once outside the cell).

Energy-emitting reporters that are useful in the systems, kits and methods of the Invention include light energy-emitting reporters, such as fluorescence- or bioluminescence-emitting reporters. Bioluminescence-emitting reporters are known to the skilled worker, and include, for example, enzymes such as luciferase or any one of its modified forms. Fluorescence-emitting reporters are also known to the skilled worker, and include, for example, GFP (Green Fluorescent Protein), EGFP (Enhanced Green Fluorescent Protein), CFP (Cyan Fluorescent Protein), YFP (Yellow Fluorescent Protein), RFP (Red Fluorescent Protein), BFP (Blue Fluorescent Protein), and their engineered variants. See, for example, light-emitting. U.S. Pat. Nos. 5,804,387, 5,360,728, 5,541,309, 5,625,048, 6,027,881, 6,054,321, 6,077,707, 6,096,865, 6,403,374.

Still other light emitting proteins that are useful as compartment-specific markers are various mutants of GFP with increased fluorescence, mutants in which the protein major excitation peak has been shifted to 490 nm with the peak emission kept at 509 nm (EGFP). Color mutants of GFP such as cyan fluorescent protect (CFP) and yellow fluorescent protein (YFP), employed for, e.g., fluorescence resonance energy transfer (FRET) experiments may be useful in this system. Genetically-encoded FRET reporters sensitive to cell signaling molecules, such as calcium or glutamate, protein, phosphorylation state, protein complementation, receptor dimerization and other processes provide highly specific optical readouts of cell activity in real time.

Also useful in the systems, kits and methods of the invention are enzymes that convert a substrate to a detectable product, such as a fluorescent product or product emitting visible light amenable for quantified measurement by standard detection equipment. Bioluminescence markers are particularly suitable in assays measuring cell viability. One exemplary bioluminescence enzyme is luciferase, which produces light upon reacting with its substrate, luciferin. Because light is emitted when luciferase is exposed to the appropriate luciferin substrate in the presence of ATP, which in general is available only in a living cell, the luciferase reaction can be used to detect living cells. Photon emission can be detected by light sensitive apparatus such as a luminometer or modified optical microscopes.

The luciferase can be from any source, including but not limited to firefly luciferase (E.C. 1.13.12.7), the Jack-O-Lantern mushroom luciferase, renilla luciferase, luciferase from any of a number of marine creatures that will be known to those of skill in the art, and click beetle luciferases, which produce different colors from the same luciferin substrate.

Bioluminescent or fluorescent compartment-specific markers are useful for a number of end points of the assay, particularly those relating to cell viability or cell proliferation, although they also can be used in other assay end points, such as cell adhesion, cell morphology changes, etc.

In certain embodiments, the marker may be a recombinantly engineered protein, including a fusion protein (such as a luciferase-fluorescent protein fusion, a fluorescent protein-luciferase fusion, fusion of two fluorescent proteins, etc., infra), or a fusion construct encoding both a luciferase and a fluorescent protein.

In certain embodiments, the cell type of interest may comprise more than one marker. For example, the cells of interest may comprise a luciferase and a fluorescent protein, either separately or in the form of a fusion protein such as a luciferase-GFP fusion or a GFP-luciferase fusion.

In certain embodiments, the accessory cells comprise a different marker than the marker in the cells of interest that can be independently monitored with respect to the marker in the cells of interest. While the methods and co-culture of the invention do not require any marker to be present in the one or more additional accessory cell types, the presence of a different independently monitorable marker in such accessory cells may be useful to track the phenotype of the accessory cells.

For example, during an assay to identify compounds that are cytotoxic to a cell type of interest, such as tumor cell in a co-culture with non-neoplastic (normal) accessory cells, a tumor-cell-specific marker provides useful information regarding the viability and other phenotypes of the tumor cells. The presence of a separate, independently monitorable marker (such as a fluorescent protein that emits at a different wavelength or color) in the accessory cells may allow one to observe the effect of compounds on normal cells. In certain embodiments, it might be desirable to select compounds that shows relatively moderate cytotoxicity towards normal cells, yet meanwhile exhibit strong selective killing of tumor cells. Similarly, in a co-culture of tumor cells with endothelial cells as the accessory cells, the ability to detect a test compound that selectively inhibits the growth of the endothelial cells may enable identification of a valuable anti-angiogenic agent, despite its relatively less-than-desirable cytotoxicity towards cancer cells.

Any art-recognized methods may be used to introduce a marker into the cell type(s) of interest (and/or the accessory cells). For example, expression vectors containing a nucleic acid encoding a subject marker polypeptide, operable linked to at least one transcriptional regulatory sequence, may be used to introduce the marker into a host cell.

“Operably linked” is intended to mean that the nucleotide sequence is linked to a regulatory sequence in a manner which allows expression of the nucleotide sequence. Regulatory sequences are art-recognized, and are selected to direct expression of the subject marker proteins. Accordingly, the term “transcriptional regulatory sequence” includes promoters, enhancers and other expression control elements. Such regulatory sequences are described in Goeddel; Gene Expression Technology: Methods in Enzylmology 185, Academic Press, San Diego, Calif. (1990). For instance, any of a wide variety of expression control sequences, sequences that control the expression of a DNA sequence when operatively linked to it, may be used in these vectors to express DNA sequences encoding the marker polypeptides of this invention. Such useful expression control sequences, include, for example, a viral long terminal repeat (LTR) sequence, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs of the present invention can also be used to deliver nucleic acids encoding the subject marker polypeptides. Thus, another aspect of the invention features expression vectors for in vitro transfection/transduction and expression of a subject marker polypeptide in particular cell types.

Expression constructs of the subject marker polypeptide may be administered in any biologically effective carrier, e.g., any formulation or composition capable of effectively delivering the recombinant gene to cells. Approaches include insertion of the subject marker gene in viral vectors including recombinant retroviruses, adenovirus, adeno-associated virus, and herpes simplex virus-1, or recombinant bacterial or eukaryotic plasmids. Viral vectors infect cells directly; plasmid DNA can be delivered with the help of, for example, cationic liposomes (lipofectin) or derivatized (e.g., antibody conjugated), poly-lysine conjugates, gramacidin S, artificial viral envelopes or other such intracellular carriers, as well as direct injection of the gene construct or CaPO₄ precipitation. One of skill in the art can readily select from amongst available vectors and methods of delivery in order to optimize expression in a particular cell type or under particular conditions.

A preferred approach for introduction of nucleic acid into a cell is by use of a viral vector containing nucleic acid, e.g., a cDNA, encoding the particular form of the polypeptide. Infection of cells with a viral vector has the advantage that a large proportion of the targeted cells can receive the nucleic acid construct. Additionally, molecules encoded within the viral vector, e.g., by a cDNA contained in the viral vector, are expressed efficiently in cells which have taken up viral vector nucleic acid.

Retrovirus vectors (including lentiviral vectors) and adeno-associated virus vectors are generally understood to be the recombinant gene delivery system of choice for the transfer of exogenous genes, due to the stable long-term expression using these vector systems. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of replication competent virus in the cell population. The development of specialized cell lines (termed “packaging cells”) which produce only replication-defective retroviruses has increased the safety and therefore utility of retroviruses for gene therapy. Use of replication-incompetent retroviruses has been well characterized for use in gene transfer for gene therapy purposes (for a review see Miller, A. D. (1990) Blood 76: 271). Thus, recombinant retrovirus can be constructed in which one or more parts of the retroviral coding sequence necessary for replication (gag, pol, env, etc) are not packaged into virions, rendering the retrovirus replication defective. The replication defective retrovirus can be used to infect a target cell by standard infection techniques, but is unable to replicate within the target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic pseudotyped retroviral systems include ψCrip, ψCre, ψ2 and ψAm. Retroviruses have been used to introduce a variety of genes into many different cell types, including neuronal cells, in vitro and/or in vivo (see for example Eglitis, et al. (1985) Science 230: 1395-1398; Danos and Mulligan (1988) Proc. Natl. Acad. Sci. USA 85: 6460-6464; Wilson et al. (1998) Proc. Natl. Acad. Sci. USA 85: 3014-3018; Armentano et al. (1990) Proc. Natl. Acad. Sci. USA 87: 6141-6145; Hober et al. (1991) Proc. Natl. Acad. Sci. USA 88: 8039-8043; Ferry et al. (1991) Proc. Natl. Acad. Sci. USA 88: 8377-8381; Chowdhury et al. (1991) Science 254: 1802-1805; van Beusechem et al. (1992) Proc. Natl. Acad. Sci. USA 89: 7640-7644; Kay et al. (1992) Human Gene Therapy 3: 641-647: Dai et al. (1992) Proc. Natl. Acad. Sci. USA 89: 10892-10895; Hwu et al. (1993) J. Immunol. 150: 4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86: 9079-9083; Julan et al. (1992) J. Gen Virol 73: 3251-3255; and Goud et al. (1983) Virology 163: 251-254); or coupling cell surface receptor ligands to the viral env proteins (Neda et al. (1991) J Biol Chem 266: 14143-14140). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic pseudotyped virus in to an amphotropic pseudotyped virus.

Moreover, use of retroviral gene delivery can be further enhanced by the use of tissue- or cell-specific transcriptional regulatory sequences which control expression of the gene of the retroviral vector.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) BioTechniques 6: 616; Rosenfeld et al (1991) Science 252: 431-434; and Rosenfeld et al. (1992) Cell 68: 143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they can be used to infect a wide variety of cell types, including airway epithelium (Rosenfeld et al. (1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Furthermore, the viral particles are relatively stable and amenable to purification and concentration, and as above, can be modified so as to affect the spectrum of infectivity.

Yet another viral vector system useful for delivery of one of the subject marker genes is the adeno-associated virus (AAV). Adeno-associated virus is a naturally occurring defective virus that requires another virus, such as an adenovirus or a herpes virus, as a helper virus for efficient replication and a productive life cycle. (For a review see Murzyczka et al. Curr. Topics in Micro. and Immunol. (1992) 158: 97-129). It is also one of the few viruses, other than lentiviruses, that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol 63: 3822-3828; and McLaughlin et al. (1989) J. Virol. 62: 1963-1973). Vectors containing as little as 300 base pairs of AA. can be packaged and can integrate and exogenous DNA packaged up to 4.5 kb. An AAV vector such as that described in Tratschin et al. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell. Biol. 4: 2072-2081; Wondisford et al. (1988) Mol. Endocrinol. 2: 32-39; Tratschin et al. (1984) J. Virol. 51: 611-619; and Flotte et al. (1993) J. Biol Chem. 268: 3781-3790).

The above cited examples of viral vectors are by no means exhaustive. Herpes-simplex viral vectors and lentiviral vectors are just two additional types of viral vectors which can be used in the present invention.

In addition to viral transfer methods, such as those illustrated above, non-viral methods can also be employed to cause expression of a subject marker polypeptide. Many non-viral methods of gene transfer rely on mechanisms used by cells for the uptake and intracellular transport of macromolecules. In certain embodiments, non-viral gene delivery systems of the present invention rely on endocytic pathways for the uptake of the subject polypeptide gene by the targeted cell. Exemplary gene delivery systems of this type include liposomal derived systems, poly-lysine conjugates, and artificial viral envelopes.

In certain embodiments, the DNA introduction may not need to be stably integrated into the host cell genome. For example, vectors with a stable extrachromosomal element (e.g., amplisome) may be used to transfect DNA into host cells.

However, in a preferred embodiment, a polynucleotide encoding the marker is stably integrated into the host genome. Preferably, the marker produces a quantifiable signal linearly and directly proportional to the number of viable cells in the host cells (e.g., the first cell type). In other embodiments, the signal merely needs to be qualitatively related to the biological function or event.

In certain embodiments, the marker produces a quantifiable signal independent of the presence or absence of the one or more additional cell type(s), and/or independent of the ratio of the cell type(s) of interest over the one or more additional cell type(s).

Markers that are not harmful to the cells and that do not appreciably affect the biological function or event of interest upon introduction, expression or detections are particularly advantageous in the systems, kits and methods of the invention. Use of such markers enables the sequential detection of the compartment of interest over the duration of the treatment. Luciferase, luciferin and fluorescent proteins are generally non-harmful to their host cells. If there is a need to verify that the mere introduction and expression of the marker itself does not appreciably alter the relevant phenotype (such as cell viability) of the host cell, the phenotype of the host cell with or without the marker may be monitored and compared (preferably before any large scale screening) to determine if there is any appreciable change.

Signals from the compartment-specific markers, such as the bioluminescent or fluorescent markers, may be detected using art-recognized means, depending on the particular type of signals generated by the marker. For example, with respect to the bioluminescent or fluorescent markers, there are numerous commercially available detectors suitable for detecting the light signal from such compartment-specific markers.

A luminometer enables highly sensitive detection for luminescent assays and thus, is particularly useful for detecting the compartment-specific bioluminescent or fluorescent markers of the invention. In certain embodiments, suitable luminometers comprise models equipped with circuitry, a CCD camera, and/or an advanced photon-counting photomultiplier tube (PMT) for producing high signal-to-noise ratios, and preferably have a detection limit of at least about 1×10-18 moles of luciferase, 1×10-19 moles of luciferase, 1×10-20 moles of luciferase, or at least about 1×10-21 moles (700 molecules) of luciferase or 3 attomole of ATP. In addition, in certain embodiments, suitable luminometers have linear dynamic ranges greater than 5, 6, 7, 8, 9, or more decades/orders of magnitude.

Most commercially available fluorometers also provide high sensitivity for detection of various fluorophores. In certain embodiments, suitable fluorometers have a detection sensitivity of about 10 ppt fluorescein, about 1 ppt fluorescein, 0.1 ppt fluorescein, or about 0.01. ppt fluorescein. In certain embodiments, suitable fluorometers have linear dynamic range of about 4, 5, 6, or 7 decades/orders of magnitude.

In certain embodiments, suitable detectors carry modules that enable detection of both bioluminescent and fluorescent signals.

The choice of specific types of useful detectors may also depend on the assay end point. For example, where the marker is a light emitter and cell viability is the endpoint, luminometers are useful, as well as other kinds of light detectors.

For instance, optical imaging detector is a highly sensitive, quantitative, non-invasive instrument suitable for use in the instant application. An exemplary optical imaging system is the IVIS® imaging system and the Living Image® Software by Xenogen, Inc. See U.S. Pat. Nos. 6,649,143, 5,650,135, 6,217,847, 6,916,462, 6,890,515 6,908,605, 7,116,354, 7,113,217, 6,922,246, 6,919,919, 6,901,279, 6,894,289, 6,775,567, 6,754,008, 6,614,452, and D469181 (all incorporated herein by reference), relating to the imaging apparatus, imaging analysis software, and methods of non-invasive, biophotonic imaging across a wide range of wavelengths, specifically including light generated by bioluminescence and fluorescence. These optical imaging detectors are capable of detecting about 10⁴ photons/sec.

In addition, a skilled artisan will appreciate that a marker of the invention producing a signal can be assayed such that determination of the signal generated is performed at more than one time point (e.g., to obtain time lapse data). In certain embodiments, determination of generated signal can be determined at intervals of microseconds, milliseconds, centiseconds, deciseconds, seconds, minutes, hours, days, and weeks or any combination thereof or encompassing similar gradations of time using other time standards. In other embodiments, the total number of measurements is determined by the skilled artisan according to well established principles of in vitro assay measurement.

Where cell morphology is the assay end point, other imaging equipment may be useful, such as the ones described below.

For example, there are many commercially available high throughput imaging equipment and software that may be used for analyzing the changes in cell morphology. Exemplary equipments include (but are not limited to) the High Content microscopes produced by Molecular Devices Corp. (CA), such as model Discovery-1™, ImageXpresss™ 5000A, ImageXpress™ Ultra, and ImageXpress™ Micro. MetaXpress™ may be used as the controlling and analysis software on all of these devices.

Data obtained from the detectors may be analyzed using a variety of art-recognized software, which may be commercially available or readily available to a person of skill in the art.

For example, MetaXpress™ features laser auto-focus, which increases scan speed, improves the using and reduces time of exposure on the sample. The laser auto-focus decreases photo-bleaching and phototoxicity concerns with live cells, and MetaXpresss™ calculates parameters based on plate dimensions and characteristics, requiring less input from users. High throughput (HT) modules are also available to accelerate the image-based screening process. These modules are optimized for the automated analysis of large compound libraries. HT modules use algorithms capable of processing image data at the speed of acquisition. MetaMorph™—the industry standard microscope automation and image analysis package—may also be used or adapted to be used in the instant invention.

If should be understood that the commercial systems described herein are merely for illustration only. Any other system that performs similar functions may be used by a person of skill in the art.

The cell co-culture of the invention may be used in numerous applications where cell-cell interaction might affect the phenotype and/or behavior of the cell type(s) of interest.

As used herein, “cell-cell interaction” includes (but are not limited to) direct physical contacts between cells. It also includes the situation where accessory cells are simply present in the microenvironment of the cell type(s) of interest. Though not in direct contact with any cell type(s) of interest, these accessory cells may affect at least one phenotype of the cell type(s) of interest by, for example, secreting cytokines or paracrine hormones, or affecting other accessory cells directly in contact with the cell type(s) of interest. Furthermore, it also applies to merely a hypothetical or potential functional modulation of the cell type(s) of interest by an accessory cell, when, for example, one is simply interested to study any potential modulatory effects a particular type of accessory cell may have on a cell type of interest (despite the fact that no documented effect is known).

Thus the cell co-culture systems, kits and methods are useful for virtually any application in which it is desired to investigate the effect of an accessory cell on a cell type of interest. Such applications are useful for elucidating biological pathways, identifying therapeutic targets, identifying therapeutic modalities and agents and for the improved prediction of efficacy in vivo. The subject co-culture system may be particularly advantageously utilized to study the effect of a test compound on cell viability, cell proliferation, cell migration, cell adhesion, cell morphology, and the like, in the presence (or absence) of one or more accessory cells.

For example, in certain embodiments, the biological activity of interest may be cell adhesion to one or move other cell types or to chemical substrates, which can be natural or synthetic, or to combinations of cells and chemical substrates, or tissue samples or fractions thereof. In certain embodiments, the biological activity of interest may be the temporal and/or spatial organization of cell morphology during interaction with one or more other cell types or chemical substrates, which can be natural or synthetic, or to combinations of cells and chemical substrates, or to tissue samples or fractions thereof. In certain embodiments, the biological activity of interest may be the status of differentiation of cells as they interact with one or more other cell types or chemical substrates, which can be natural or synthetic, or with combinations of cells and chemical substrates, or with tissue samples or fractions thereof.

In certain embodiments, the biological activity of interest may be the proliferation and viability of bacterial or fungal cells as they are allowed to grow in culture media containing one or more other cell types, chemical substrates, or tissue samples or fractions thereof.

Typically, accessory cells are inoculated into multi-well microliter plates in, e.g., 100 μL, at plating densities ranging from 1,000 to 150,000 cells/well (typically 10,000 cells/well) depending on the doubling time of individual cell lines. For cell plating of adherent cells (e.g. stromal cells), the microliter plate cultures are incubated at 37° C., 5% CO₂ for 8-24 hrs to allow cell attachment. The second cell type(s) of interest (e.g., tumor cells) are then plated at various cell densities (typically 1,500-20,000 cell/well). The cells are then treated with test compounds for 1-3 days before signal detection.

According to one method of the invention, a cell co-culture of the invention, i.e., a co-culture comprising a cellular compartment of interest comprising cells stably transfected with a marker whose detection is specific for that compartment and further comprising a cellular accessory compartment, is contacted with one or more compounds of interest, preferably (but not necessarily) in high throughput format; and comparing the signal generated by the compartment-specific marker with and without the contacting step, wherein a statistically significant change in the signal from the co-culture contacted with the compound of interest compared to the signal in the absence of the compound is indicative that the compound modulates at least one biological activity in the cells of interest in the presence of accessory cells. In a particularly advantageous embodiment, the compound of interest is a potential therapeutic agent. The effect of the compound may be reducing an undesirable activity or increasing a desirable cellular activity. More particularly, the method may be used to test the cytotoxic, cytostatic/cytoreductive ability of one or more compounds, biological agents, or other stimuli in the presence of tumor accessory cells, or, conversely, the ability of compounds, biological agents or other stimuli to trigger tumor cell proliferation, increased viability or resistance to other agents. In such a method, the compartment-specific marker may enable the detection and/or quantification of viable cells. According to the invention, the above-described method may be expanded to include multiple cell types of interest, multiple accessory cell types, multiple concentrations of the test compounds and multiple test compounds or combinations of compounds in a highly multiplexed experiment.

Those of skill in the art will appreciate that essentially any parameters may be adjusted according to specific designs of the experiment, such as concentrations of the drugs or cells, the duration of the experiment, culturing conditions (such as media pH, substrate or growth factors added, temperature of culturing) and the like.

In an exemplary use of this embodiment, compounds may be pre-screened using isolated tumor cell lines and compounds that show promise in the pre-screening are further tested its the co-culture system of the invention. Compounds that perform better in the presence of the accessory cells may be preferred for further research and development, while those perform significantly worse in the presence of the accessory cells may command lower research and development priority.

The invention also provides a method for detecting the effect of an accessory cell on a cell type of interest. According to this method, a cell type of interest that is stably transfected with a compartment-specific marker is contacted with one or more compounds of interest, both in the presence and in the absence of an accessory cell compartment and the signal from the compartment of interest with and without the accessory compartment is compared. A statistically significant change in the signal from the compartment of interest in the presence of accessory cells compared to that produced in the absence of accessory cells indicates that the candidate compound can affect the cellular biological function or event, and the impact of the cell co-culture on the effect of the candidate compound on the cellular biological function or event of interest (e.g., whether the candidate compound is more effective or at least as effective in the presence of the accessory cells). In certain embodiments of the invention, the cell co-culture and the second cell culture are contacted by the candidate compound(s) at substantially the same time, or at different times.

According to this embodiment of the invention, cell type(s) of interest in the co-culture are compared to cell type(s) of interest alone (without the accessory cells) with respect to their responses to a candidate compound. The advantage of this method is that it allows one to assess the effect of accessory cells on the response of the cell type(s) of interest towards the candidate compound. It also allows one to specifically identify those compounds or agents that perform better (or worse) in the presence of accessory cells. Compounds that perform better in the presence of the accessory cells may be preferred for further research and development, while those perform significantly worse in the presence of the accessory cells may command lower research and developmental priority.

Alternatively, if a second compound is known to inhibit the function of the accessory cell or kills the accessory cell, a combination therapy may be used to antagonize the accessory cell function, thus further potentiating the effect of the candidate compound. Conversely, if a second compound is known to enhance the function of the accessory cell, a combination therapy may be used to stimulate the accessory cell function, thus further potentiating the effect of the candidate compound. If no such second compound is known, a search/screen for such compound new research may be pursued for such combination therapy.

This embodiment of the method may also be used to identify compounds or agents that do not seem to perform differently in the presence or absence of accessory cells. For example, when a library of candidate compounds are being screened, the majority of the compounds in the library are not expected to effectively kill the cell type(s) of interest (or accessory cells). Thus the reading for the marker in the majority of the assays is expected to be roughly the same, indicating no effect on cell viability. For the few compounds that actually reduce cell viability (but show no difference in efficacy either in the presence or absence of accessory cells), the readings from both the co-culture and the pure cell culture (without accessory cells) will be roughly the same, but both readings would be lower than the average reading from the other assays ran in parallel. Thus in some embodiments, a statistically significant decrease in the signal from the cell co-culture and/or the second cell culture compared to the average signal is indicative that the candidate compound is useful for affecting the cellular biological function or event.

These embodiments of the invention are not limited to screening for compounds. In fact, the methods of the invention are generally applicable to screens for any therapeutic candidate of interest, including various non-compound-based treatment methods.

Thus for example, the invention also provides a screening method (preferably a high throughput screening method) for identifying a treatment useful for affecting a cellular biological function or event, the method comprising: (1) providing a cell co-culture of the invention; (2) subjecting the cell co-culture to said treatment, preferably (but not necessarily) in high throughput format; (3) monitoring and comparing a signal generated by the marker from the cell co-culture before and after the treatment; wherein a statistically significant change in the signal after the treatment compared to that before the treatment is indicative that the treatment is useful for affecting the cellular biological function or event. Exemplary treatments include radiation therapy, cellular immunotherapy, such as exposure to immune effector cells (e.g., T-cells, Natural Killer cells, etc.) or other cells participating in the cellular arm of immune responses), therapy utilizing light or heat, etc.

In any embodiment of the invention, if a library of candidate compounds are used, the library may comprise synthetic compounds, natural compounds, or a mixture thereof.

In certain embodiments in which two or more compounds are used, the cell co-culture system of the invention may be contacted with a mixture or “cocktail” of the compound, by the compounds separately, or both. Where the co-culture is contacted with the two or more compounds separately, the contacting can be simultaneous or sequential. Where the contacting is sequential, the cell co-culture system may be pre-treated by a first test compound or compounds, followed by a second batch of one or more other compounds. Optionally, the first batch of compounds are first removed (e.g., by washing away with buffers) before the second batch of compounds are added.

In certain embodiments where it is desired to test various treatment regimens comprising multiple therapeutic agents or modalities, treatment of the co-cultures of the invention with one or more test compounds may be combined with any of the non-compound-based treatments described herein, with the treatments occurring in any desired order or simultaneously.

In any embodiment of the invention, at least one compound in the library is tested at two or more different concentrations. This may be beneficial because the same compound may have different effective ranges of concentrations against different cell types or against the same cell type under different conditions. In certain embodiments, the two or more different concentrations spans at least about 2, 3, 4, 5, 6, 7, 8, 9, 10 or more orders of magnitude in terms of test compound concentration. In the initial experiments, a wider range of concentrations (such as 3-5 concentrations over 10 orders of magnitude) may be used, while in further experiments, more data points might be spread over a smaller concentration range. In certain embodiments, the medium concentration tested is the concentration closest to known effective concentration in human for the compound or structurally similar compounds. Those of skill in the art are familiar with selecting concentrations that are useful in the methods of the invention.

In any embodiment of the invention, the candidate compounds may be from a polypeptide library, an antibody library, a small molecule library, a polynucleotide library, or a mixture thereof. “Small molecule” as used herein includes molecules with a molecular weigh of no more than 50 Da, 100 Da, 200 Da, 500 Da, 1 kDa, 2 kDa, or 5 kDa. “Polynucleotide library” may include antisense oligonucleotides, an siRNA library, a cDNA library, a genomic DNA library, etc.

In certain embodiments, the candidate compounds may comprise one or more anti-cancer drugs which may include, but are not limited to, the following: methotrexate, busulfan, thioguanine, 6-mercaptopurine, nitrogen mustard, guanaxole, R-methylformamide, actinomycin D, chlorambucil, thiadsazole, thio-tepa, DON, melphalan, borterzomib, dexamethasone, triethylenemelamine, hexamethylenemelanime, gallium nitrate, 5-fluorouracil, thymidine, delta-1-testololactone, mitramycin, pipobroman, cyclophosphamide, mitomycin C, 5-FUDR, hydroxyurea, methyl-GAG, uracil nitrogen mustard, O6-methylguanine, o,p′-DDD, DTIC, vinblastine sulfate, IMPY, porfiromycin, chromomycin, cytosine arabinoside, vincristine sulfate, thalicarpine, B-TGDR, A-TGDR, fluorodopan, D-tetrandrine, procarbazine, CCNU, daunorobicin (daunomycin), S-trityl-L-cysteine, streptozoticin, methyl-CCNU, PCNU, hexamethylenebisacetamide, 3HP, Yoshi-864, 5-azacytidine, cytembena, 5HP, L-asparaginase, iphosphamide, pentamethylmelamine, diglycoaldehyde, cisplatin, VM-26 (teniposide), doxorubicin (Adriamycin), bleomycin, paclitaxel (Taxol), dichloroallyl lawsone, 3-deazauridine, 5-azadeoxycytidine, triazinate, ICRF-159, diahydrogalatitol, indicine N-oxide, rifamycin SV, piperazinedione, soluble Baker's Antifol, emofolin sodium, anguidine, VP-16 (etoposide), homoharringtonine, hycanthone, pyrazofurin, cyelocytidine, ftorafur, hydrazine sulfate, L-alanosine, maytansine, neocarzinostatin, AT-125 (acivicin), rubidazone, bruceantin, asaley, ICRP-187, spirohydantoin mustard, chlorozotocin, tamoxifen, AZQ, spirogermanium, aclacinomcein A, 2′-deoxycoformycin, PALA, rapamycin, largomycin, CBDCA (carboplatin), m-AMSA (amsacrine), caracemide, CHIP, 3-deazaguanine, dihydro-5-azacytidine, glycoxalic acid, deoxydoxorubicin, N,N-dibenzyldaunomycin, menogaril, (carboxyphthalato) platinum, pyrrolizine dicarbamate, triciribine phosphate, ARA AC, trimethyltrimethylolmelamine, mitindomide, 8Cl-cyc-AMP, tiazofurin, pyrimidine-5-glycodialdehyde, flavoneacetic acid ester, teroxirone, DHAD (mitoxantrone), aphidicolin glycinate, L-cysteine analogue, acodazole hydrochloride, amonafide, fludarabine phosphate, SR2555 (nitroimidazole), batracylin, nitroestrone, pibenzimol hydrochloride, bactobolin, didemnin B, L-buthionine sulfoximine, phyllanthoside, hepsulfam, macbecin II, rhizoxin, tetrocarcin A sodium salt, merbarone, bisantrene hydrochloride, penclomedine, clomesone, chloroquinoxaline sulfonamide, bryostatin, fostriecin, dihydrolenperone, piperazine alkylator, flavoneacetic acid, cyclodisone, pancratiastatin, oxanthrazole, 4-ipomeanol, trimetrexate, mitozolamide, morpholino-ADR, anthrapyrazole, deoxyspergualin, cyanomorpholino-ADR, pyrazine diazohydroxide, tetraplatin, pyrazoloacridine, bispyridocarbazolium DMS, DUP785 (brequinar), cyclopentenylcytosine, ARA-6-MP, BCNU, echinomycin, carmethizole, topotecan, and MX2 HCl.

In high throughput embodiments of the invention, any format may be used, as long as it is scalable and suitable for high throughput detection system. By way of illustration, the high throughput format may comprise plates or other containers with any number of wells, such as six-well, 12-well, 24-well, 48-well, 96-well, 384-well, or 1536-well, etc. As a skilled artisan will appreciate, the choice of format will depend on the specific assays (e.g., certain assays may preferably be carried out in larger wells or smaller wells). Regardless of well sizes, sample volume, or other assay parameters, it is a feature of the invention that the methods of the invention are in a scalable format that can be carried out in large numbers or high throughput with ease, although individual experiments or assays using the methods need not always be carried out in high throughput.

In embodiments utilizing a fluorescent signal, any suitable detection means for detecting such signal may be used, such as a plate reader. Data received form the detector (e.g., the plate reader), such as relative or absolute light intensity, may be recorded and stored electronically to allow further data processing, analysis and comparison, preferably by any suitable software means.

In any embodiment of the invention, the method may further comprise determining the ability of the identified candidate compound to affect the activity of the marker, wherein an identified candidate compound not substantially affecting the activity of the marker is useful for affecting the cellular biological function or event. This may be useful for eliminating certain rare false positive hits, where identified compounds in fact affect the expression or activity of luciferase (e.g., either by enhancing or suppressing the bioluminescent readout) without affecting the cell viability. Alternatively, when coupled with conventional assays, such as the MTT assay, these false positive effects would be apparent.

In another aspect, the invention also provides a kit, which may be used to practice The methods of the invention. The kit may comprise: (1) a vector encoding a marker amenable to high-throughput screening; and (2) a medium suitable for co-culturing two or more cell types. In certain embodiments, the kit can additionally comprise (3) plates for culturing; and (4) detection reagents for measurement.

Any suitable vector described herein may be used for the kit of the invention. Preferably, the vector can mediate the introduction of subject marker into the host cell, such as a cancer cell, and preferably stably expressing the marker by, for example, stably integrating the marker-encoding polynucleotide into the host genome.

In certain embodiments, the vector is a plasmid, a retroviral vector, or a lentiviral vector.

In certain embodiments, the kit further comprises means for introducing the vector into cells, including (but are not limited to): transfection/infection reagents or helper cells, reagents for selecting stably-transfected cells (if a drug-resistant selectable marker is used), etc.

The choice of the medium depends on the specific cell types to be co-cultured. It may not be the optimal medium for growing either cell type alone. For example, if one cell type grows optimally in medium A, while the other cell type grows best in medium B, a series of mixtures of media A and B with different percentages of medium A (10%, 15%, 20%, 25%, 30%, 35%, 40%, 45%, 50%, 55%, 60%, 65%, 70%, 75%, 80%, 85%, 90%, 95%, 99% or any range or combination therein) may be tested on cell co-culture to obtain the optimal medium for cell co-culture.

Alternatively, individual components of one medium (such as serum, percentage, etc.) may be adjusted. For example, if cancer cell grows best in 5% serum, while normal cells grows best in 10% serum, a medium with 6%, 7%, 8%, 9% serum may be tested and optimized for survival and growth of both cell types.

An exemplary cancer cell medium is RPMI 1640 medium containing 5-10% fetal bovine serum, and optionally 2 mM L-glutamine. The medium may also be supplemented with antibiotics, such as 100 U/ml penicillin and 100 μg/ml streptomycin.

In certain embodiments, the kit may further comprise at least one cell isolation and/or culturing means, including (but are not limited to): surgical instruments (e.g., biopsy needles, surgical knives, etc.); enzymes for tissue digestion (such as Dispase II (Roche Molecular Biochemicals), trypsin, collagenase, etc.); cell/tissue handling instruments (such as scalpel blades, meshes, etc.); and/or tissue culture vessels, etc.

In a related aspect, the kit of the invention may comprise: (1) a vector encoding a marker amenable to high-throughput screening; and, (2) a conditioned medium from the accessory cells.

In certain embodiments, the secreted factors from the accessory cells may contribute to the majority of the effects seen in cell co-culture, while cell-cell contact between the first cell type of interest and the accessory cell may be of secondary importance. In these embodiments, conditioned medium may be harvested from the accessory cell culture, and used to grow the first cell type of interest (e.g., cancer cells).

The kits of the invention may be especially useful in the field of cancer drug screening, where certain frequently used cancer cell lines, such as one or more of the NIH/NCI panel of 60 cancer cell lines used for initial lead drug screening, may be packaged with one or more matching accessory cells (such as one or more normal cells present in the microenvironment in which the cancer cells grow in vivo) or conditioned media thereof for use in the methods of the invention.

Thus another aspect of the invention provides a kit comprising: (1) one or more cancer cells; and (2) one or more normal cells that interact with the cancer cells in vivo, or conditioned media thereof.

In certain embodiments, the cancer cells may be from a primary site cancer or a secondary, metastatic cancer. The cancer cells may be freshly isolated from the tumor tissues, or such isolated tumor cells in the first few generations of enduring.

### 2. Use of the Invention in Cancer

The cell co-culture system, methods, and kits of the invention may be used in a variety of biological contexts, for a wide range of uses, such as screening for compounds that affects the biological function of a cell type of interest in the presence of one or more accessory cell type(s).

To illustrate the general inventive concept further, described herein is an exemplary embodiment of the invention concerning a specific type of applicable biological system—one involving tumor cells and certain accessory cells from the in vivo tumor milieu. When used in tumor biology, one aspect of the instant invention fills the void in cancer drug development by establishing models that allow the quantification of tumor cell viability both in the presence and absence of non-neoplastic co-cultured cell populations and under experimental settings amenable to high-throughput applications.

Historically, the early stages of anti-cancer drag development involve high-throughput screening of large libraries of compounds for potential in vitro activity against tumor cell lines. In these screening modalities, tumor cells are studied in conventional cell culture systems in isolation from any other cell types which make up the tumor microenvironment. Many studies have contrasted the remarkable in vitro activity of conventional and investigational anti-cancer agents with their typically less impressive activity in clinical trials.

Applicants have realized that this discrepancy is due, at least in part, to the protection that non-neoplastic accessory cells can confer to the tumor cell population in the tumor microenvironment. Tumor cell interactions with the tissue microenvironment, support the proliferation and survival of malignant cells, even rescuing them from systemic anti-cancer therapies. Multiple myeloma (MM) cells, for example, which are highly responsive to dexamethasone (Dex) in conventional monolayer cultures, become significantly less responsive to Dex treatment in the presence of bone marrow stromal cells (BMSCs).

Conventional cultures of tumor cells do not take into account the possibility that accessory cells in the microenvironment can attenuate tumor cell responsiveness to any number of anti-neoplastic agents. As a result, the classical high-throughput assays for anti-cancer drug screening, which are also based on conventional in vitro cultures of isolated tumor cells, may both overestimate and underestimate the anti-tumor activity of a tested drug.

By way of example, a drug that may be very active against tumor cells in isolation in vitro, but against which tumor cells develop resistance when they are co-cultured with stromal cells, would score as a promising candidate in a conventional screening program, but would be likely not to perform well in pre-clinical studies in orthotopic in vivo models and eventually in clinical trials. In such a case, the conventional assays would overestimate the activity of the particular anticancer drug. This might account, at least in part, for several cases of anti-cancer compounds that showed promising activity in early conventional in vitro drug sensitivity studies, but showed significantly lower levels of activity in subsequent clinical trials.

Conversely, it is plausible that certain agents may exhibit modest, if any, substantial direct single-agent activity against tumor cells, but may be able to abrogate the tumor-stromal interaction and/or suppress the activity of pathways that mediate its functional consequences on tumor cells. Such compounds would not be scored as positive hits in a conventional screening program. In other words, novel anti-cancer agents that target the interaction between the tumor cells and cells in the tumor microenvironment would not be identified from conventional drug screening assays, thereby limiting the potential for their further development. However, such agents could in principle be very useful drugs for the management of various cancers. This last principle is exemplified by thalidomide and lenalidomide, which have modest single-agent in vitro anti-MM activity, but which are potent anti-MM agents in vivo because, among other mechanisms of their action, they can inhibit several aspects of the interactions of MM cells with non-malignant accessory cells of their local microenvironment, such as BMSCs. A conventional drug screening program might not identify thalidomide or lenalidomide as promising anti-MM agents, but assays which take into account tumor cell-accessory cell interactions might uncover an in vitro efficacy signal sufficient to lead to their further consideration for pre-clinical studies and eventual clinical trials.

Despite the importance and advantages of screening that takes into consideration of the tumor-microenvironment interaction, most available tumor cell cytotoxicity/viability assays (such as the MTT assay, Alamar Blue assay, LDH release assays, etc.) do not permit discrimination between cell types in a co-culture system. On the other hand, existing assays that test anti-tumor activity in the context of tumor-stromal interactions (e.g., ³H-thymidine incorporation or flow cytometry-based assays) are not conducive to high-throughput application for a variety of technical and conceptual reasons (such as handling large quantities of radioactive materials, slow and complicated operator-dependent screening steps, and/or prohibitively expensive reagent costs, etc.).

The instant invention solves these problems by providing cell co-cultures (e.g., tumor cells co-cultured with accessory cells) for use in various reliable high-throughput drug sensitivity assays, which to a large extent reduce or eliminate some discordant results in the oncological field between in vitro drug screening and in vivo anti-tumor activity. Applicants have established an experimental system whereby tumor cells engineered to stably express a bioluminescence-related enzyme, such as luciferase, are co-cultured with accessory cells of the local tumor milieu (e.g., BMSCs). Using the co-culture and methods of the invention, compounds with anti-tumor activity attenuated by cells from the tumor milieu are detected earlier on in the process of drug development. In addition, drug sensitivity testing in tumor cell-accessory cell co-cultures allows the identification of compounds that do not have direct anti-tumor properties, but that may abrogate tumor-stromal interactions or its functional sequelae. Such compounds would not give an efficacy signal in conventional screening programs, which test tumor cells in isolation, and which, due to their limitations, may have over-looked many useful compounds by failing to recognize their merits for further potential potential clinical studies.

Thus the co-culture systems, kits and methods of the subject invention, in which tumor cells are exposed to potential therapeutic treatments in co-culture with accessory cells from the tumor's normal in vivo milieu, are an ideal pathophysiologically relevant model system for cancer drug screening.

Tumor cells that are useful in the systems, kits and methods of the invention may be from an established cancer cell line, which might be adapted for in vitro culturing, or from a primary tissue sample. The tumor cells can be from any mammal including mouse, rat, pig, goat, cow, monkeys or humans. Cells from any tumor type may be used in the instant invention. The tumor may be a solid tumor, or a hematological tumor/cancer. Exemplary solid tumors include (but are not limited to): sarcoma or carcinoma of the bone, cartilage, soft tissue, smooth or skeletal muscle, CMS (brain and spinal cord), Peripheral Nervous System (PNS), head and neck, esophagus, stomach, small or large intestine, colon, rectum, GI tract, skin, liven, pancreas, spleen, lung, heart, thyroid, endocrine or exocrine glands, kidney, adrenals, prostate, testis, breast, ovary, uterus, cervix, etc. Exemplary hematological/blood cancers include (but are not limited to): leukemia (such as adult or childhood Acute Lymphoblastic Leukemia (ALL), adult or childhood Acute Myeloid Leukemia (AML), Chronic Lymphocytic Leukemia (CLL), Chronic Myelogenous Leukemia (CML), Hairy Cell Leukemia), lymphoma (such as AIDS-Related Lymphoma, adult or childhood Hodgkin's Lymphoma, adult or childhood Non-Hodgkin's Lymphoma, T-Cell Lymphoma, or Cutaneous Lymphoma), myeloproliferative disorders (e.g., polycythemia vera, essential thrombocythemia, chronic idiopathic myelofibrosis), myelodysplastic syndromes, e.g. essential thrombocytemia, polycythemia vera, or Multiple Myeloma (MM).

Tumor cells from non-malignant tumors are also useful in the systems, kits, and methods of the invention, and may include, for example, adenoma, chondroma, enchondroma, fibroma, myoma, myxoma, incidentaloma, benign, neurinoma, osteoblastoma, osteochondroma, osteoma, papillary tumor, papillary tumour, papilloma, villoma, etc.

The selection of accessory cells depends in part on the tumor cell type of interest and the nature of the experiment. Any cell type whose effect on tumor cells is desired to be studied can be used. Accessory cells may be non-tumor cells that occur in the tumor milieu in vivo or cells that do not occur in the tumor milieu. Cells that are useful as accessory cells may be from the same species as the tumor cells or from a different species, may be from a different stage of tumor development than the tumor cells of interest or may be non-tumor cells. Useful accessory cells include cells from any organ or tissue in which a tumor can occur including but not limited to bone marrow stromal cells, mesenchymal cells, fibroblasts, adipocytes, bone cells, endothelial cells, pericytes, immune cells, liver cells, kidney cells, prostate cells, ovarian cells, cervical cells, cells of the central nervous system including brain and spinal cord neurons, muscle cells, stomach cells, esophageal cells, cells that interact with the tumor cell in vivo, and cells that may directly or indirectly affect cancer cell behavior. In fact, cells from any tissue or organ where a tumor (malignant or benign) can arise may be useful as accessory cells.

In the case of tumor cells from a primary tissue sample, the tumor cells may be from a primary tumor site (e.g., from a tumor mat originates in a tissue or organ, in contrast to a tumor that results from metastases from a distant location). In the case of a primary melanoma, accessory cells may comprise karetinocytes, fibroblasts, or other skin cells that normally occur in the milieu of the melanocytes in vivo.

Alternatively, the tumor cell may be from a metastatic site, such as the lung. In this case, the accessory cells may comprise lung cells that form the microenvironment of the metastatic melanoma.

The magnitude of protection against chemotherapeutics or other agents conferred by accessory cells may differ, depending on the particular tumor cell line tested and anti-tumor agent tested. For example, KU812F cells develop stromal-derived resistance to Ara-C and imatinib, while K562 do not exhibit such phenotype. These results highlight the importance of testing the impact of stromal-tumor interactions on drug responsiveness, in diverse combinations of tumor types, stromal cell types and drugs classes. Thus in certain embodiments, the screening methods of the invention include all possible permutations of the highly multiplexed sets of experimental conditions, including treatment concentration, treatment duration and cell number, and using sufficient replicates using the subject rapid and sensitive techniques in high-throughput applications.

The systems, kits and methods of the instant invention also are useful to re-examine the currently available or developing drugs or drug candidate. By comparing results in the systems and methods of the invention to results in traditional single culture experiments, one may better predict the clinical response to these drugs or drug candidates due to the micro-environment effects on tumor cells and the ability of agents to overcome these effects than is possible using current screening modalities.

In fact many of the results Applicants have obtained through this high-throughput screen have suggested an explanation to the observed clinical relapse of CML patients to imatinib mesylate, which could be due, in large part, to the protective effects of the stromal microenvironment.

Thus in one aspect, the invention also provides a method of identifying a compound that overcomes accessory cell-mediated tumor cell resistance to an anti-tumor compound, the method comprising: (1) contacting the cell co-culture system of the invention with a test compound and the anti-tumor compound, wherein the one cellular compartment of interest comprises a tumor cell, and a second cellular compartment comprises non-malignant accessory cells, and, wherein the accessory cells confer accessory cell-mediated tumor cell resistance to the anti-tumor compound; (2) detecting the signal generated by the compartment-specific marker front the cell co-culture system in the presence and absence of the test compound; wherein a statistically significant change in the signal with the test compound compared to that without the test compound is indicative that the candidate compound overcomes accessory cell-mediated tumor cell resistance to the anti-tumor drug.

In certain embodiments, the method further comprises verifying that the identified test compound does not substantially affect the signal generated by the compartment-specific marker from the cell co-culture system, in the absence of the anti-tumor drug.

In this aspect of the invention, in a cell co-culture system of tumor cells and accessory cells, a given anti-tumor drug of interest may not be effective against the tumor cells. This is frequently seen in chemotherapy, where an anti-cancer drug works well for one type of cancer but not another, or works well in the initial treatment, but works poorly in treating a relapse disease. At least in some cases, tumor resistance to the drug is mediated through accessory cells in the tumor's microenvironment in vivo. The methods of the invention allows one to test and identify a test compound that may overcome this accessory cell-mediate tumor resistance, by rendering the tumor cells sensitive to drug treatment.

In certain cases, the test compound itself may be acting directly on the tumor cells in the presence of the accessory cell. In this case, the newly identified test compound itself is effective in treating a tumor resistant to a known anti-tumor drug. Alternatively, the test compound may have no effect on the tumor cell per se, but either makes the anti-cancer drug more potent, or antagonizes a function of the accessory cell critical for the accessory cell-mediate tumor resistance, or both. This later mechanism may be distinguished from the former mechanism by further testing and verifying that the identified test compound does not substantially affect the signal generated by the compartment-specific marker (in the tumor cell compartment) from the cell co-culture system in the absence of the anti-tumor drug.

Libraries of tumor cell lines expressing a compartment-specific maker, such as luciferase, for use in the systems, kits and method of the invention are another aspect of the invention that would allow the screening of various co-culture combinations.

Further, the systems, kits and methods are useful for evaluating the effect of accessory cells on tumor cell responsiveness to non-pharmacological forms of cytoreductive non-surgical interventions, including radiation therapy, photodynamic therapy (see review by Stables and Ash, Cancer Treat Rev. 21(4): 311-23, 1995), cellular vaccine therapy, cellular immune therapy (such as Donor Lymphocyte Infusion or DLI), etc.

For example, photodynamic therapy (PDT) is a well-investigated locoregional cancer treatment in which a systemically administered photosensitizer fixer is activated locally by illuminating the diseased tissue with light of a suitable wavelength. Thus the cell co-culture system of the invention may be used, for example, to test various photosensitizers in combination with the light activator, against a panel of different tumor cells, in order to determine whether a particular photosensitizer is effective against any particular tumor cells.

This instant invention represents a new approach for evaluation of drug sensitivity of tumor cells for several reasons: (1) it involves cells (e.g., tumor cells) with a stable marker (e.g., a luciferase and/or a GFP); (2) the measurement of viability of the (tumor) cells does not require cell lysis or incubation with exogenous enzymes (such as luciferase); (3) this technique can be used both in conventional culture systems, where tumor cells are cultured in isolation from any other cell types, and in settings where tumor cells are cultured with stromal cells or other potential accessory cells of the local tumor micro-environment; (4) as a result of (3), the described invention is particularly suitable to ask the question whether an anti-cancer drug of interest shows significant reduction of its activity when its target cancer cells are interacting with normal cells of their milieu, a feature which is now considered to be an ominous sign for the clinical potential of an anti-cancer drug and which cannot be assessed by conventional anti-cancer drug screening assays; (5) as a result of (3), the described invention is particularly suitable to ask the question whether a therapeutic agent of interest shows significant increase of its activity against its target cancer cells when the latter are interacting with normal cells of their milieu, a favorable feature which is now considered to be a desired property for a potential anti-cancer drug, and which also cannot be readily assessed by conventional anti-cancer drug screening assays; (6) the combined effect of (4) and (5) renders this invention a significant advance for the screening and further study of cancer therapies over the conventional screening methods; (7) as a result of (2) this technique is amenable to time-lapse measurements of compartment-specific biological responses, such as tumor cell viability, thus allowing the serial measurement of readout in the same culture across many time points, a feature that offers greater density of data collection from each experimental setup. Furthermore, the simple nature of the assay (such as read-out from an energy-emitting reporter) is particularly suitable for automation and large scale/high throughput screenings.

### 3. Other Exemplary Embodiments (Non-Cancer Cell Use)

The cell co-culture system, kits and methods also may advantageously be used in the area of AIDS study and/or drug development. For example, the target of HIV-1 virus, CD4⁺ T cells, may be labeled by a compartment-specific marker (such as luciferase or any of the other markers described herein). Any of a wide variety of cell types may be used as accessory cells in this system, including (but are not limited to) stromal cells, fibroblasts, B-lymphocytes, Natural Killer (NK) cells, macrophages, monocytes, neutrophils, eosinophils, basophils, mast cells, dendritic cells, etc. Viruses, such as HIV-1, and various anti-AIDS medicaments may also be present in the co-culture system. In certain embodiments, the compartment-specific marker may be present in the virus, which infects the cell compartment of interest and expresses the marker in the infected cells.

One exemplary use of such a system is a method to screen for anti-AIDS drugs, such as those that can stimulate the accessory cells to confer resistance of T-cells against viral infection and/or prevent the demise of CD4⁺ T cells because of the HIV virus. Alternatively, for any potential drug candidate, one or more accessory cells may be tested to determine if their presence or absence affects drug efficacy, and or their effects on viral infection. As those skilled in the art will appreciate, any host cell/virus system may also be similarly used in the systems, kits, and methods of the invention.

In another exemplary embodiment, the invention may be used in the area of study and/or drug development for inflammatory disorders. For example, depending on the specific design of the experiment, any one or more immune cell types, such as T- and B-lymphocytes, Natural Killer (NK) cells, macrophages, monocytes, neutrophils, eosinophils, basophils, mast cells, dendritic cells, etc. may be cell compartments of interest labeled by compartment-specific marker(s). Conversely, any other immune cell types, such as T- and B-lymphocytes, Natural Killer (NK) cells, macrophages, monocytes, neutrophils, eosinophils, basophils, mast cells, dendritic cells, etc. may be accessory cells in these experiments. In addition, any cells from any tissues that may be involved in inflammation, including any by-stander cells, may be used as accessory cells. Cytokines, blocking antibodies, hormones, pharmaceuticals, or any other biological agents of interest may be added to such a co-culture system to study, for example, how certain agents may affect inflammatory reaction of given cell co-culture, which candidate agent (from a screen) may affect inflammatory reaction of given cell co-culture, or which accessory cells may positively or negatively affect the efficacy of an anti-inflammatory drug in a given inflammatory disease model (cell co-culture).

Inflammatory diseases in which this embodiment may be used include, but are not limited to, asthma, allergic rhinitis, atopic dermatitis, autoimmune conditions, such as systemic lupus erythematosus, scleroderma/systemic sclerosis, polymyositis/dermatomyositis, and Sjogren's syndrome, as well as cutaneous vascilitides, Crohn's disease, ulcerative colitis, pancreatitis, hepatitis, gastritis, enteritis, etc.

In yet another exemplary embodiment, an infectious agent, such as a pathogenic bacterial cell, a fungal cell, a parasitic cell, etc. may be labeled by a compartment-specific marker and used as the cell type of interest. Host immune system cells, such as those described herein, may be used as accessory cells. Any anti-bacterial agents (such as antibiotics), anti-fungal agents, anti-parasitic agents, cytokines, hormones, second messengers, etc. may be included in the cell co-culture system. Such a system may be used to study, for example, antibiotic resistance by bacteria, and how such resistance can be overcome or, conversely, triggered by any accessory cells or agents that stimulates the accessory cells, and to identify therapeutic compounds.

These embodiments, together with the tumor embodiments, are merely a few illustrative uses of the instant invention. The co-culture systems, kits, and methods of the invention can readily be used in any other complex biological systems involving two or more cell types.

### EXAMPLES

The general concept of the invention having been described, the section below provides several working examples to further illustrate the cell co-culture system of the instant invention. The Examples are merely for illustration and are not intended to be limiting in any respect.

In certain embodiments, the described “in vitro CSBLI” (“in vitro Compartment-Specific BioLuminescence Imaging”) assay allows tumor cells to be detected, irrespective of the presence or absence of other non-neoplastic cells, because of selective emission, upon luciferin administration in the culture, of bioluminescence by the luciferase-positive viable tumor cells, but not from dead tumor cells or from luciferase-negative stromal cells.

### Example I

### In Vitro Compartment-Specific Bioluminescence Imaging (CS-BLI) Signal from Stable Luciferase-Expressing Tumor Cells Correlates with the Number of Viable Tumor Cells

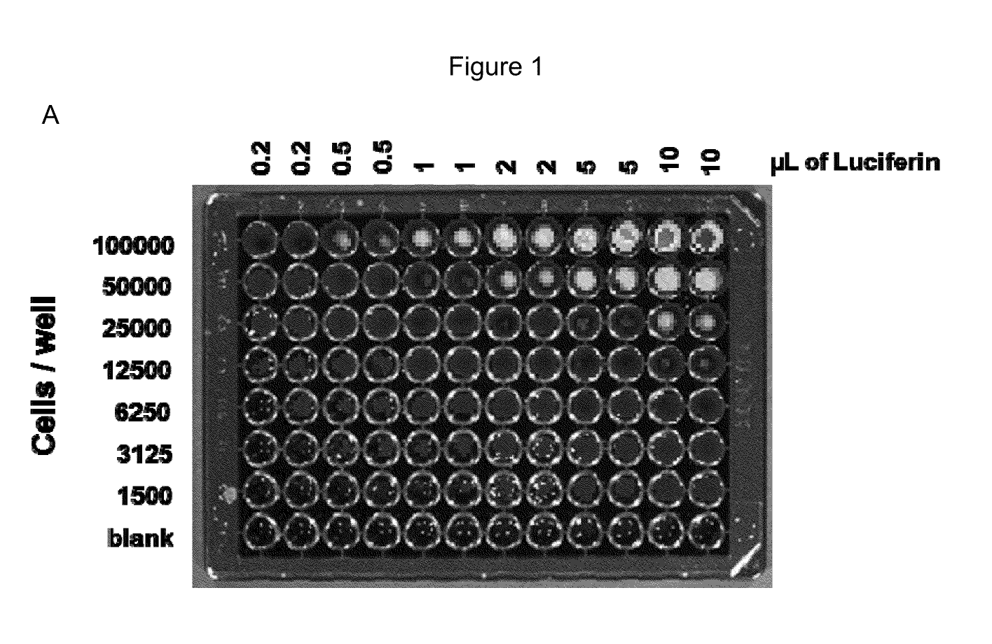

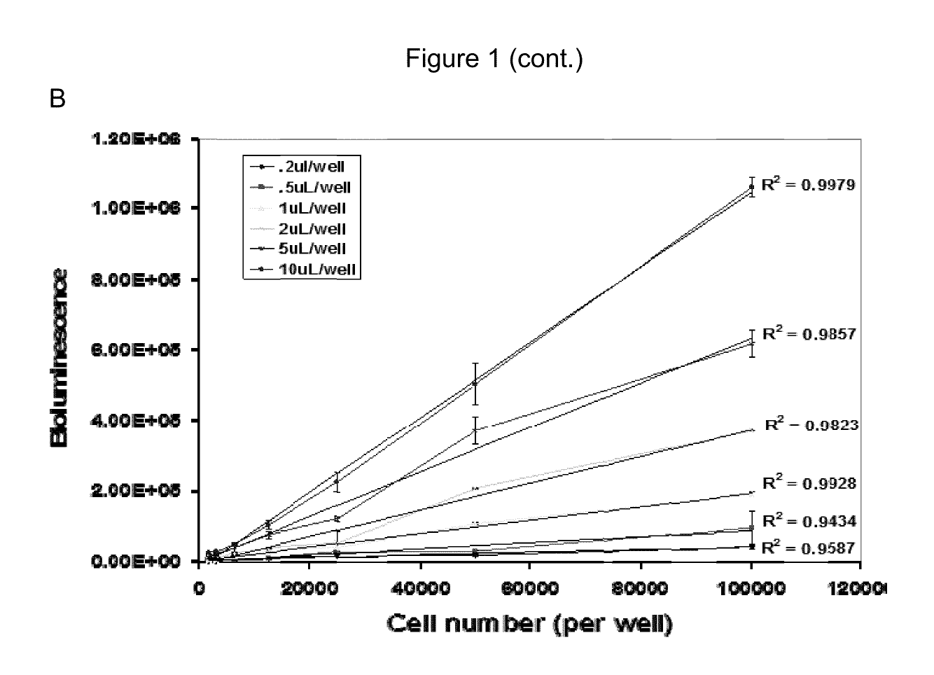

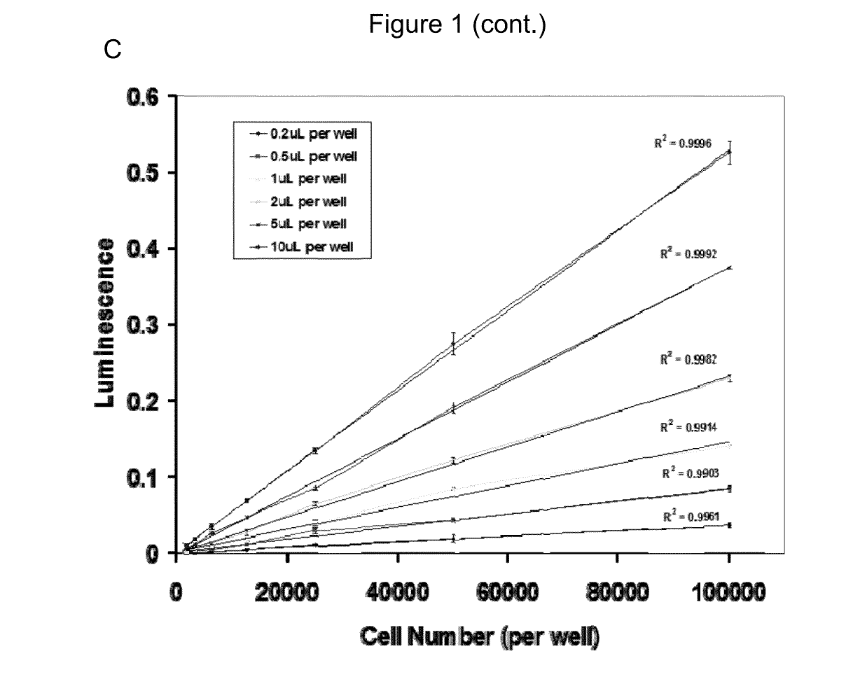

The human multiple myeloma (MM) cell line MM-1S-GFP-Luc (which has been engineered to stably express a fusion construct of lucifease-GFP) was grown in RPMI 1640 medium (BioWhittaker) supplemented with 100 U/ml penicillin, 100 μg/ml streptomycin, and 10% fetal bovine serum (FBS; GIBCO/BRL, Gaithersburg, Md.), and plated at increasing cell concentrations and increasing doses of luciferin substrate. Specifically, MM1S-GFP-luc cells were plated in optical 96-well plates (Fisher Scientific) at 1,500-100,00 cells per well in triplicate at a volume of 100 μL per well. Luciferin (7.5 mg/ml; Xenogen Corp, Alameda, Calif.) was added at the volume stated in each experiment. Cell viability and the precise cell counts were established by Trypan blue exclusion assay immediately before plating of the cells. Compartment-specific bioluminescence emitted by individual wells of these plates was measured with two different bioluminescence imaging modalities, namely a Xenogen IVIS® Imaging System (FIGS. 1A and 1B) and a standard luminometer plate reader—Luminoskan luminometer (Labsystems, MA) (FIG. 1C). The results with each method were analyzed for the linearity of the bioluminescent signal vs. cell number using the Living Image® software (Xenogen Corp, Alameda Calif.).

The compartment-specific bioluminescent signals detected with both techniques had a statistically significant linear correlation with the number of viable cells in each well (with p-values<0.001 and R² value>0.94 for each luciferin concentration using the Xenogen imaging system and >0.99 using the luminometer plate reader system).

In addition, MM-1S-GFP-Luc cells were plated at increasing cell numbers in 96-well optical plates pre-seeded with HS-5 bone marrow stromal cells, and were compared to identical cell numbers in the absence of stromal cells. In this experiment, the HS-5 (American Type Culture Collection, ATCC, Manassas, Va.) stromal cells were propagated in DMEM medium with 100 U/ml penicillin, 100 μg/ml streptomycin and 10% FBS. Co-cultures of the malignant cell line MM-1S-GFP/Luc with the HS-5 stromal cells were grown in RPMI 1640 medium with 10% PBS, 100 U/ml penicillin and 100 μg/ml streptomycin. To prepare the co-culture, HS-5 stromal cells were plated at a density of 10,000 cells per well in optical 96-well plates, and were incubated for 24 hrs to allow for Attachment. Tumor cell line stably expressing luciferase (e.g., MM-1S-GFP/Luc) was plated at 1,500-100,000 cells per well at a volume of 100 μL per well. Cells were treated immediately following plating, and incubated for 24-72 hrs as indicated. Five microliters of luciferin (7.5 mg/mL stock) was added to cultures, mixed, and incubated at room temperature for 10 min. Samples were read using a Labsystems Luminoskan luminometer. The result again showed statistically significant linear correlation between bioluminescent signal and viable cell number (FIG. 1D).

Therefore, these experiments demonstrate that compartment-specific bioluminescent signal correlates linearly with tumor cell number, both in the presence and absence of stromal cells.

### Example II

### In Vitro CS-BLI-Based Detection of Viable Tumor Cells Provides Results Consistent with Conventional Survival Assays

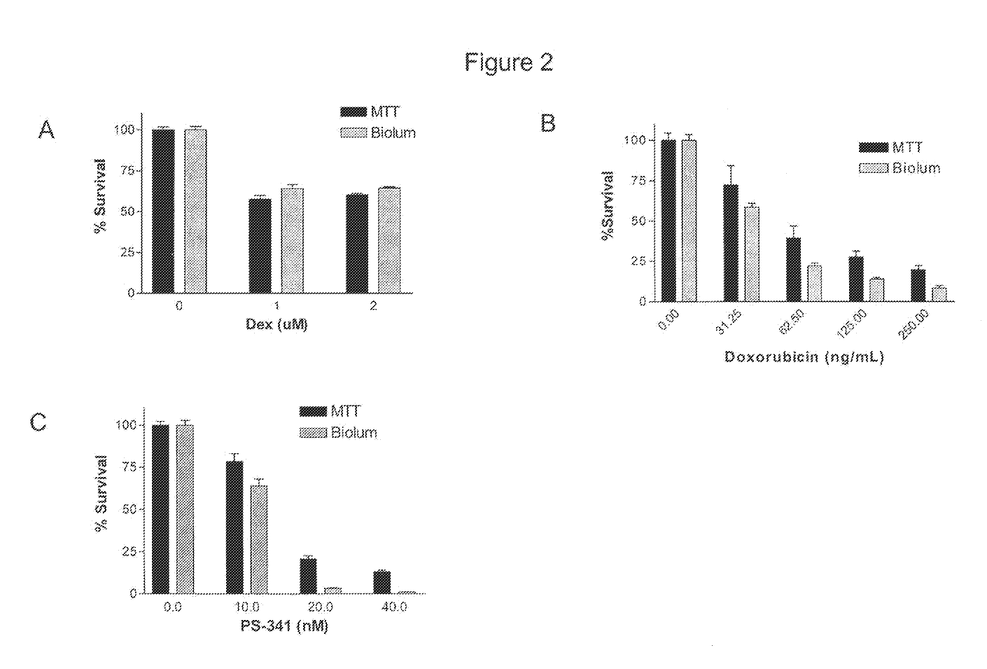

In this experiment, Applicants evaluated whether compartment-specific bioluminescence imaging provides results consistent with conventional techniques, such as MTT assay, for detection of viable tumor cells in assessment of their response to various therapeutics. MM.1S-GFP-luc cells were treated, in the absence of stromal cells, with the anti-tumor agents Dexamethasone (Dex, at 1 or 2 μM), Doxorubicin (Doxo, at 31.25, 62.5, 125, or 250 ng/mL), and bortezomib (Velcade™, formerly known as PS-341, at 10, 20, or 40 nM). Results obtained with bioluminescence detection were consistent with MTT data (FIG. 2).

In the MTT cell survival assay, viability of cells treated with anti-tumor agents was assessed by measuring 3-(4,5-dimethylthiazol-2-yl)-2,5-diphenyl tetrasodium bromide (MTT, Chemicon International, Temecula, Calif.) dye absorbance. Cells were pulsed with 1:10 the culture volume of 5 mg/ml MTT to each well for the last 4 hrs of the indicated duration of culture, followed by addition of a 0.04 N HCl solution in isopropanol (added at a volume 1.5-3 fold of the of the original culture volume). MTT crystals were dissolved by vigorous pipetting. Absorbance was measured at 570 nm (references wavelength of 630 nm) using a spectrophotometer (Molecular Devices Corp., Sunnyvale Calif.).

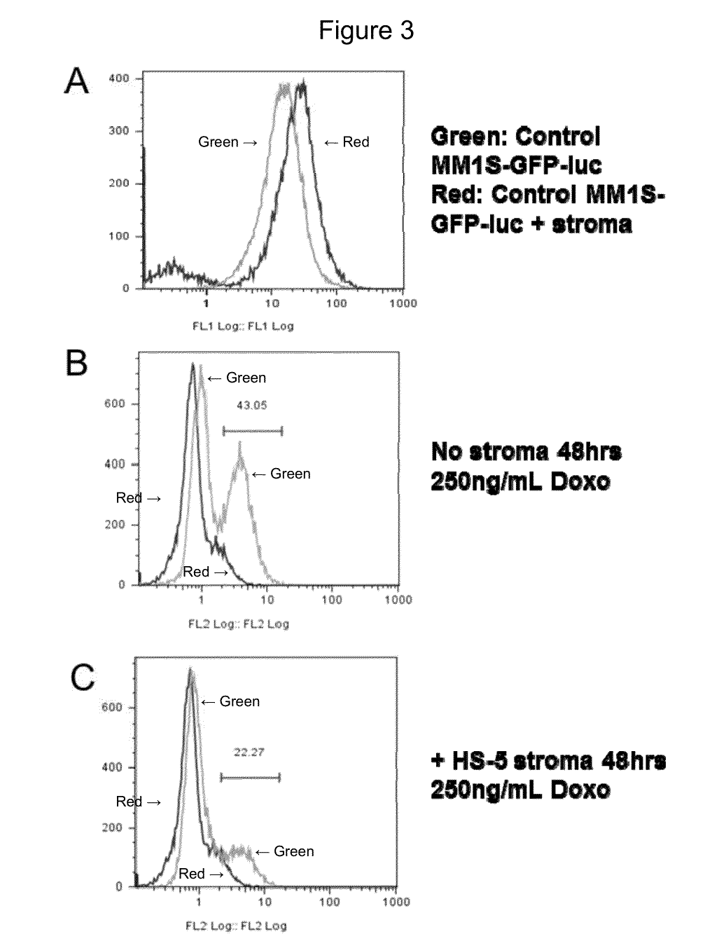

Applicants also evaluated the anti-tumor effects of these drugs in the presence of stromal cells, and compared the results obtained with in vitro compartment-specific bioluminescence vs. flow cytometric evaluation of drug-induced cell death. Specifically, luciferase-expressing MM cells were treated in vitro with Doxo (250 ng/mL) or vehicle, in the absence vs. presence of bone marrow stromal cells (BMSCs). For flow cytometry-based drug sensitivity testing in tumor-stromal co-culture, tumor cell viability in the presence vs. absence of stromal cells was evaluated by flow cytometry. Specifically, after incubation with drug or vehicle, MM-1S-GFP-luc myeloma cells were stained with Apo2.7 (BD Biosciences), to detect apoptotic cells. GFP positive myeloma cells were gated to distinguish them from GFP negative stromal cells in the co-cultures. The ratio of Apo2.7 positive cells in the GFP⁺ compartment of the drug-treated condition vs. the vehicle-treated condition provided a quantified expression of viable cells in response to drug treatment. The results showed that luciferase-GFP positive MM-1S cells (MM-1S-GFP/Luc) treated with Doxo had decreased expression of Apo2.7 after 48 hrs, when these cells were co-cultured with HS-5 stromal cells compared to cells cultured in the absence of stroma (FIG. 3). These flow cytometric results were consistent with results obtained with CS-BLI based applications as described in Example III.

### Example III

### In Vitro CS-BLI-Based Assays Identify Stroma-Mediated Protection or Sensitization of MM Tumor Cells against Various Treatments

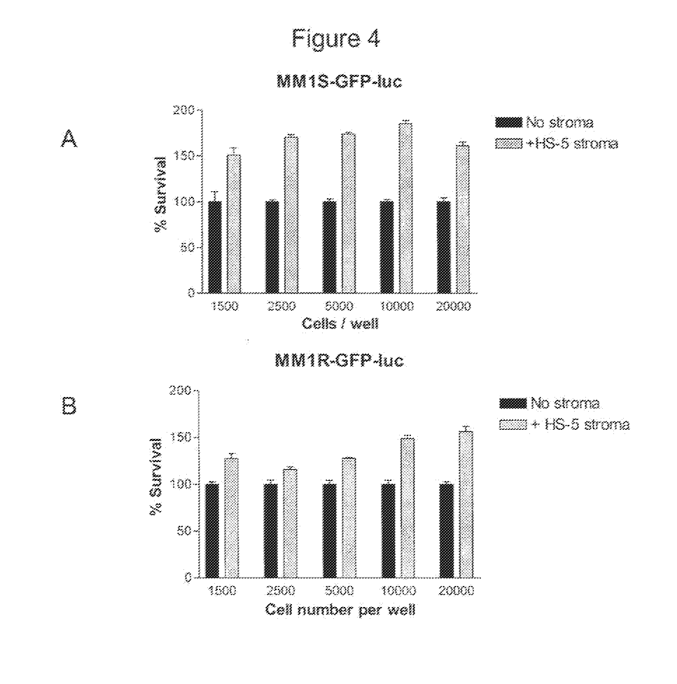

To further validate the utility of compartment-specific bioluminescence assays in this setting, Applicants compared the response of MM cells to various anti-cancer therapies in the presence vs. absence of various stromal cells. Culture conditions were identical to these described in the examples above unless otherwise indicated herein or in the figures. Applicants observed that co-culture with BMSCs increased the population of viable MM cells following incubation without drug in both MM-1S-GFP-Luc and MM-1R-GFP-Luc cells (FIGS. 4A and 4B).

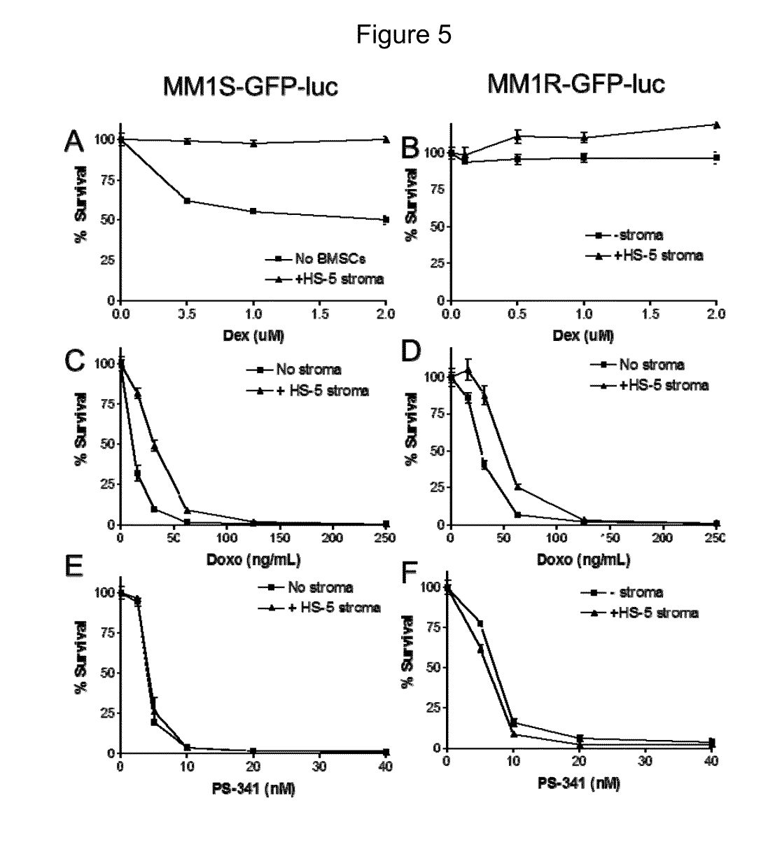

In addition, co-culture with BMSCs attenuated the responses of MM-1S and MM-1R cells to Dex (0-2.0 μM) and Doxo (0-250 ng/mL) treatment (FIGS. 5A-5D); but did not abrogate the responsiveness of MM cells to the proteasome inhibitor bortexomib (0-40 nM) (FIGS. 5E and 5F).

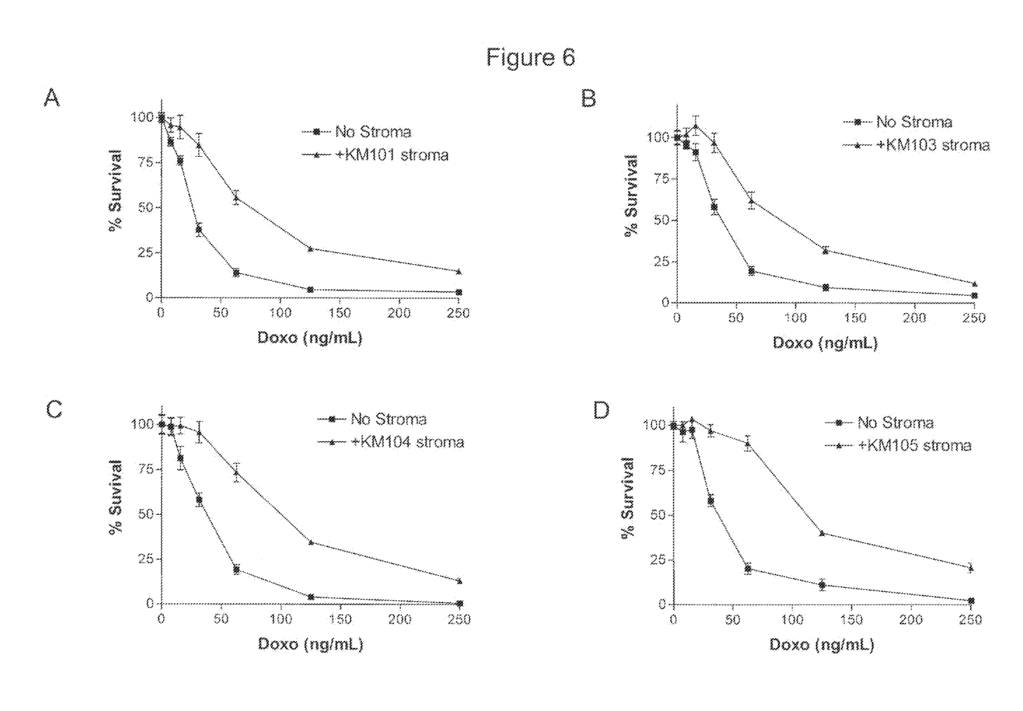

Importantly, Applicants observed that the protective effects of stromal cells on MM cells against Doxo was recapitulated with the use of different BMSC lines, such as KM101, KM103, KM104 and KM105 stromal lines (FIGS. 6A-6D, with 0-250 ng/mL of Doxo). Here, the KM101, KM103, KM104 and KM105 stromal cells were propagated, in DMEM medium with 100 U/ml penicillin, 100 μg/ml streptomycin and 10% FBS. Co-cultures of malignant cell lines with the stromal cells were grown in RPMI 1640 medium with 10% FBS, 100 U/ml penicillin and 100 μg/ml streptomycin.

Similar protective effects against 0-250 ng/mL Doxo were also observed when MM1S-GFP-luc or MM1R-GFP-luc cells were co-cultured with either NCH3T3 cells (mouse fibroblasts) or HBK293 cells (human embryonic kidney cells) (data not shown).

Similarly, the presence of HS-5 stroma also conferred protection to MM1S-GFP-luc or MM1R-GFP-luc myeloma cells against the Hedgehog inhibitor 11-keto-cyclopamine (0-8 μM) (data not shown).

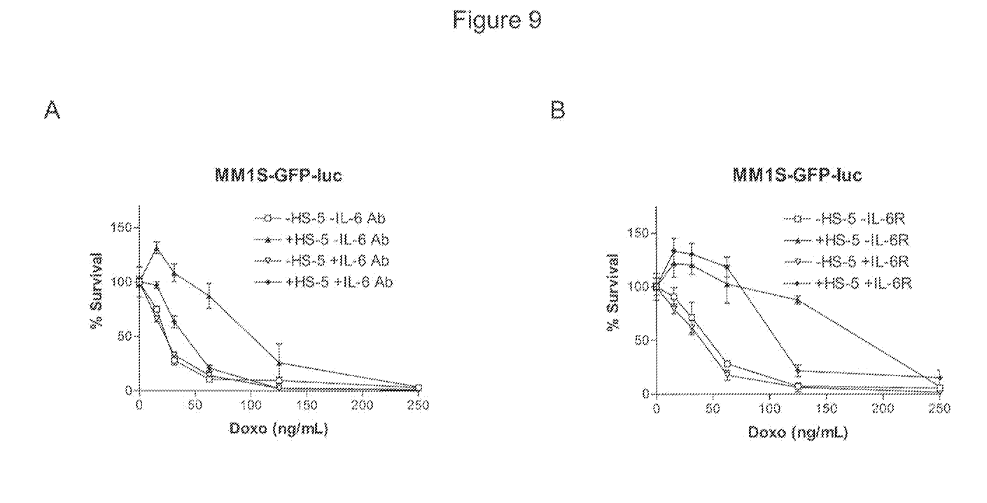

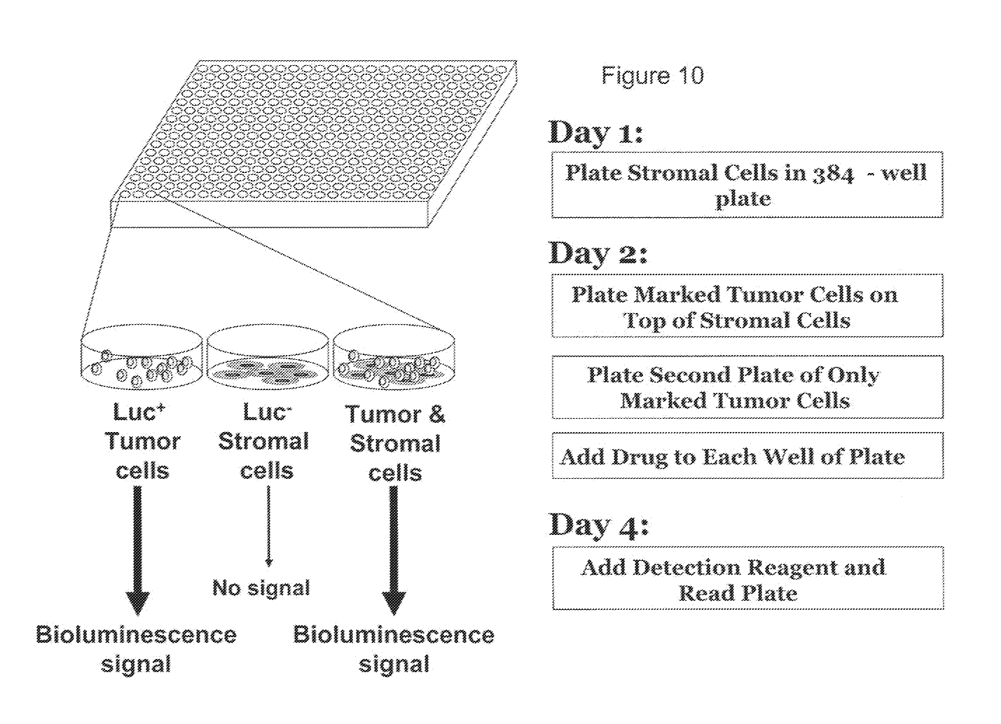

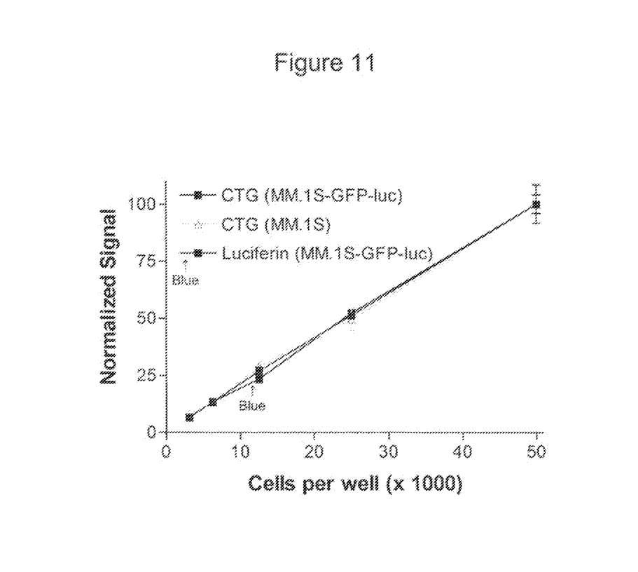

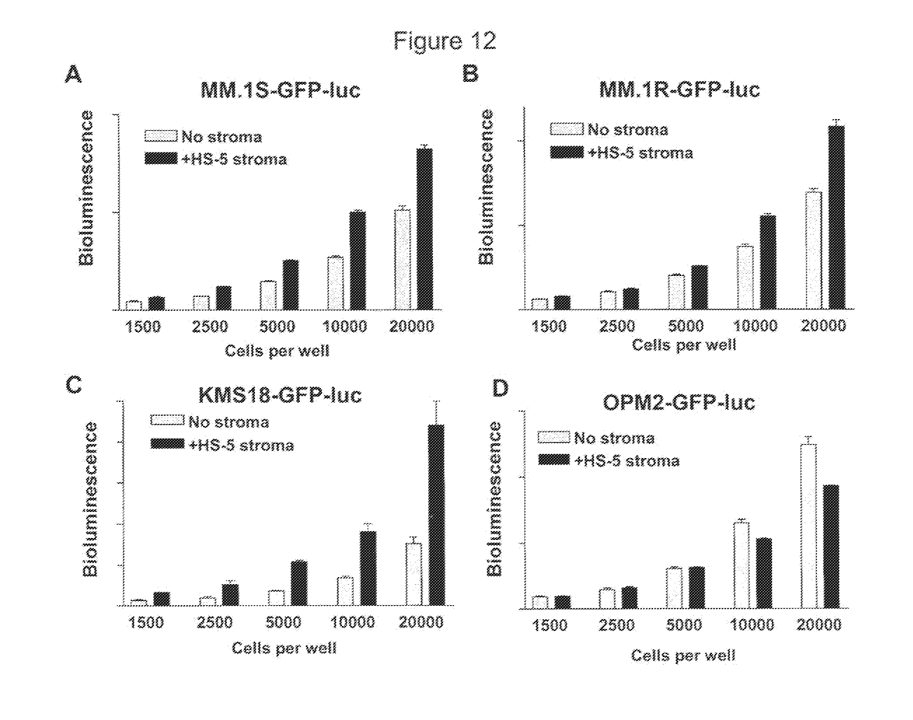

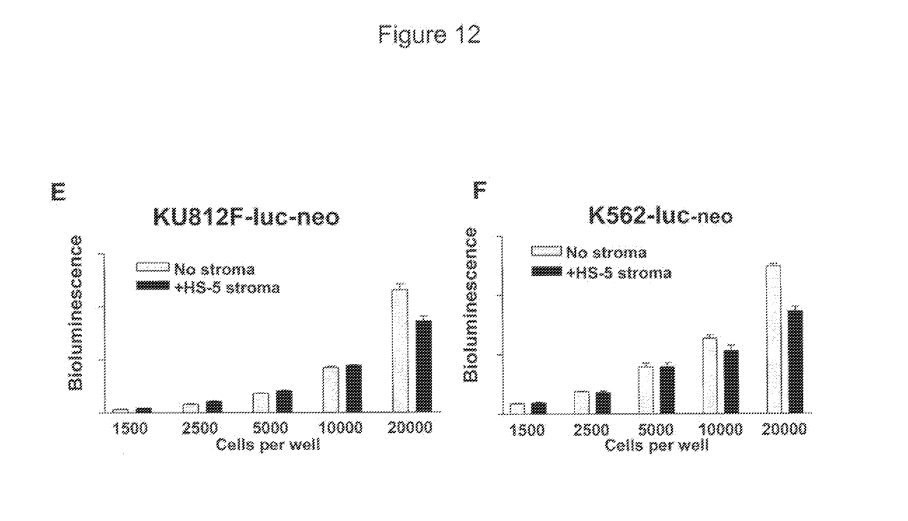

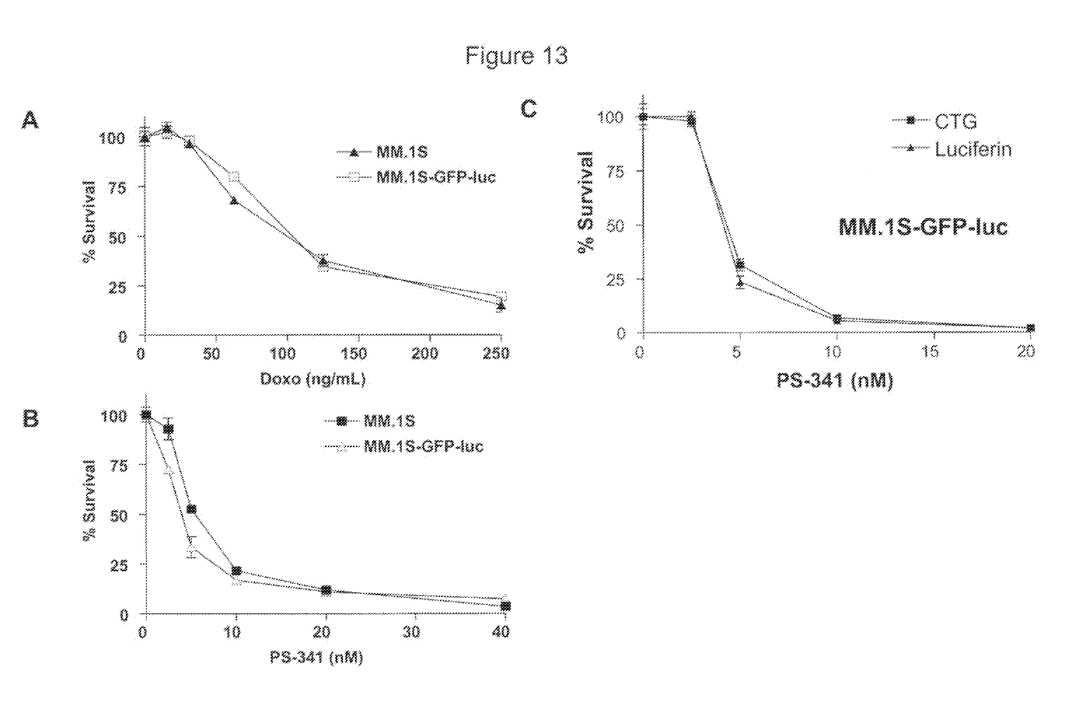

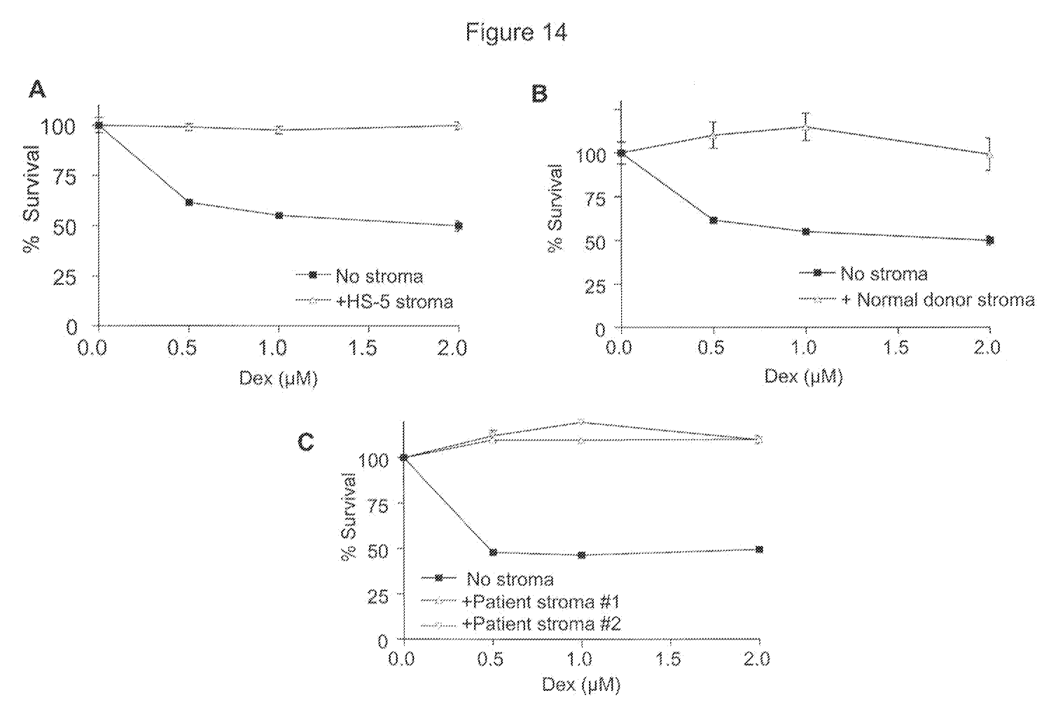

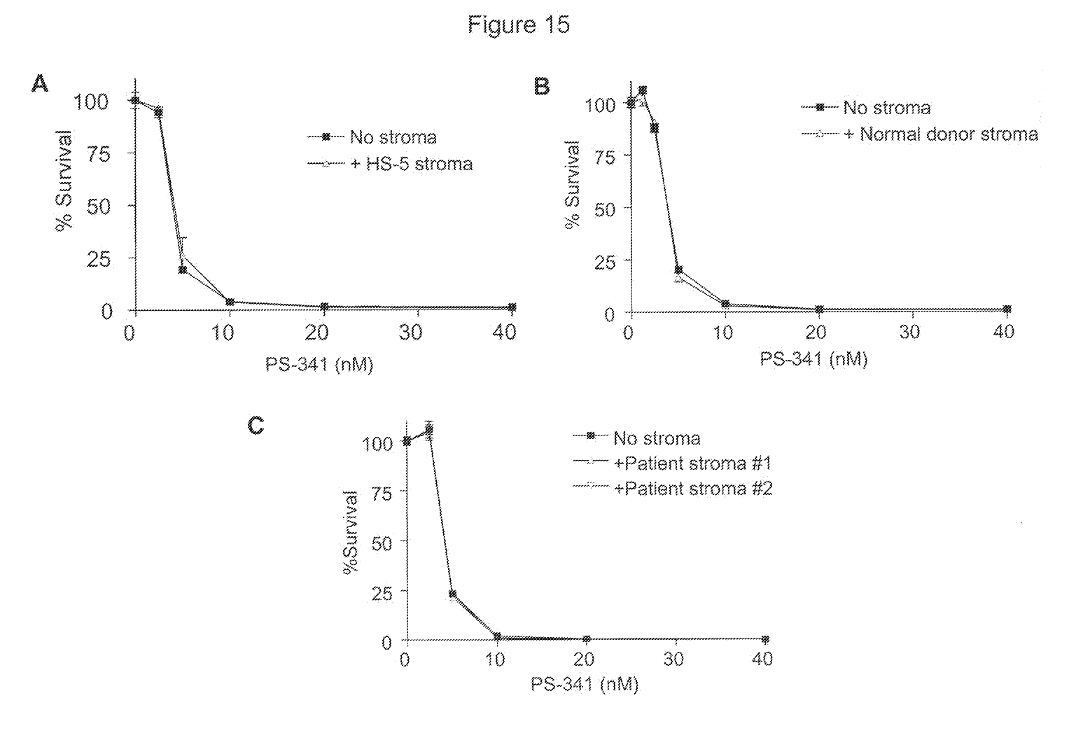

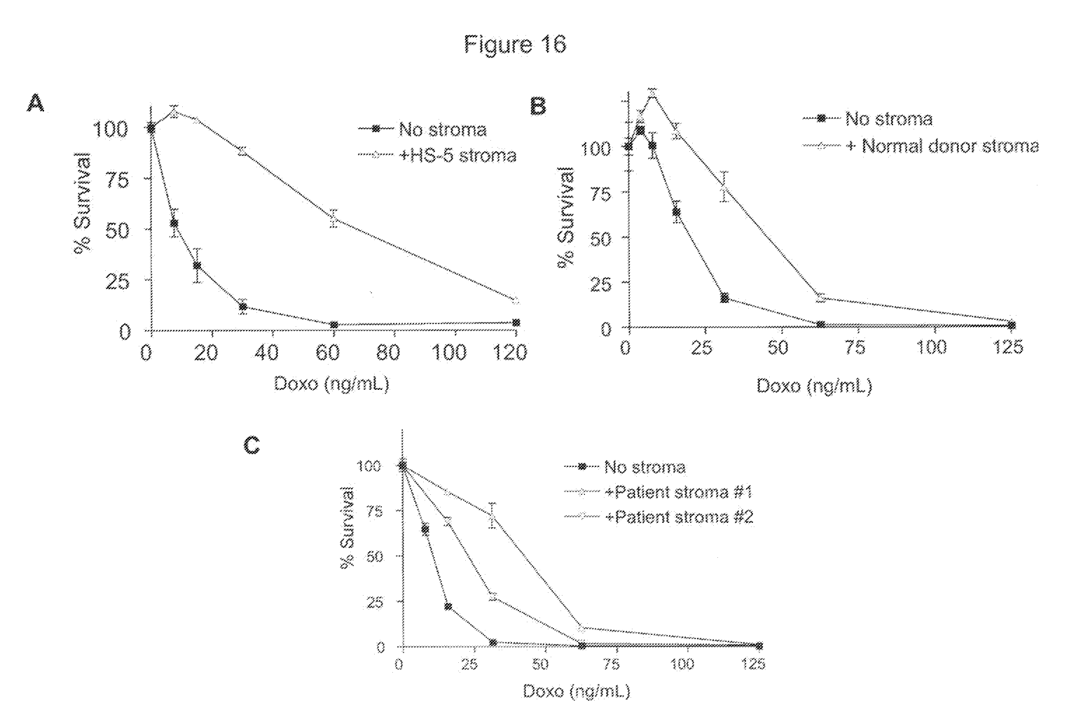

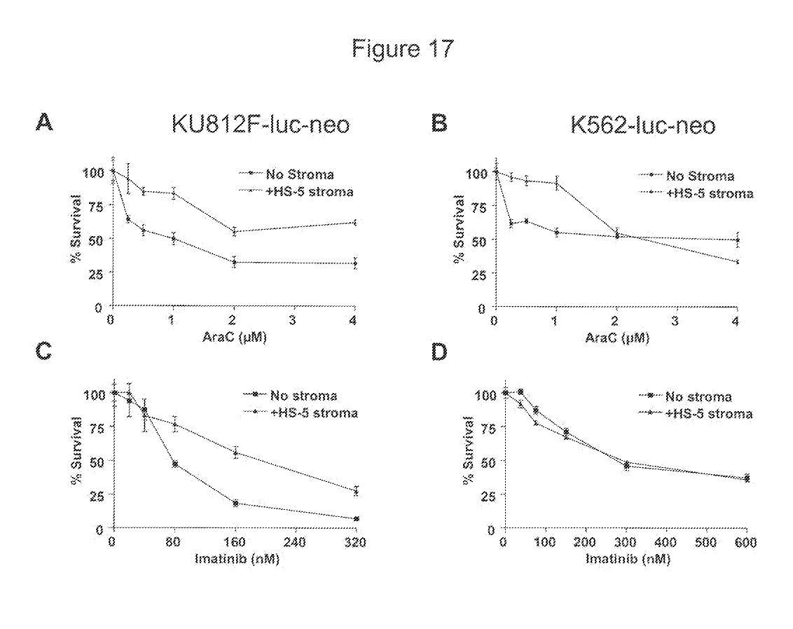

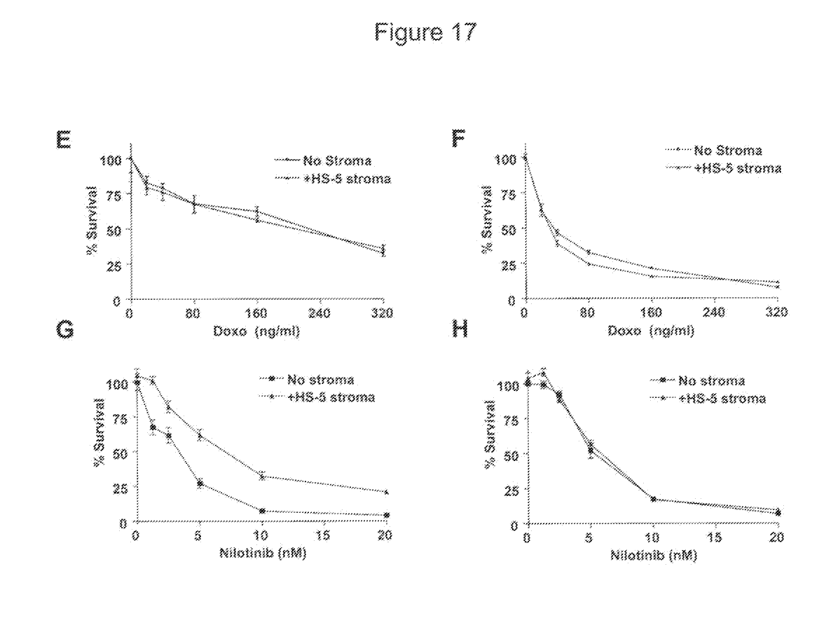

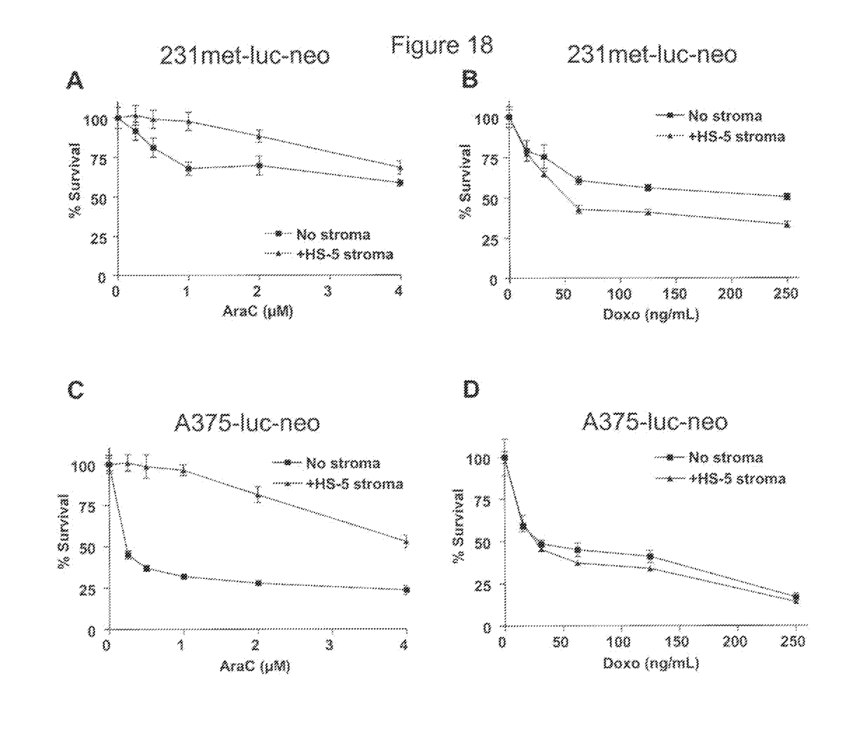

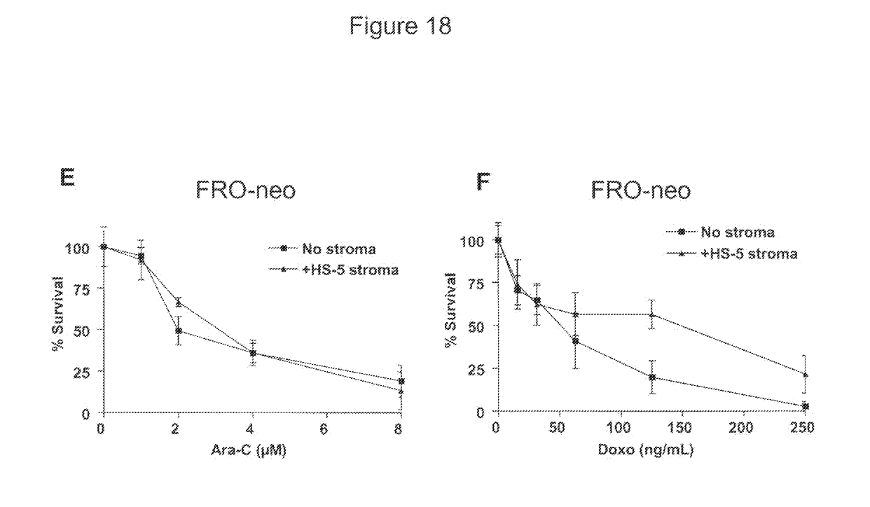

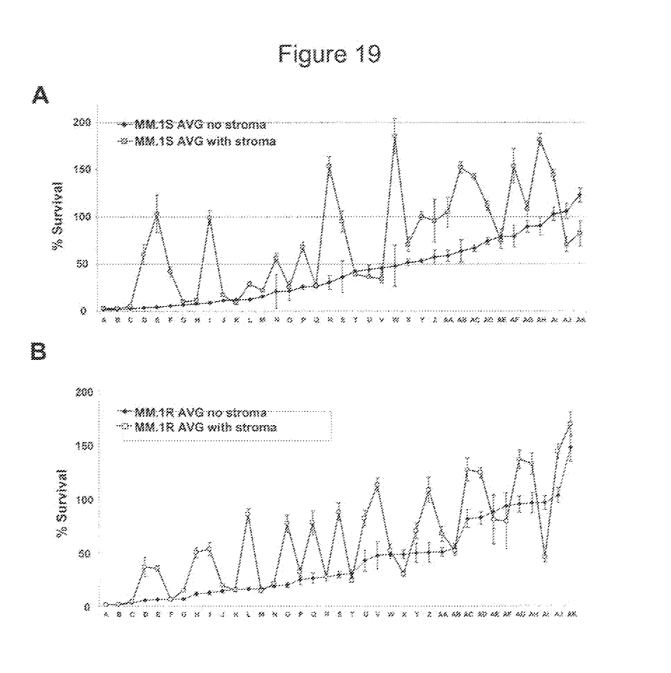

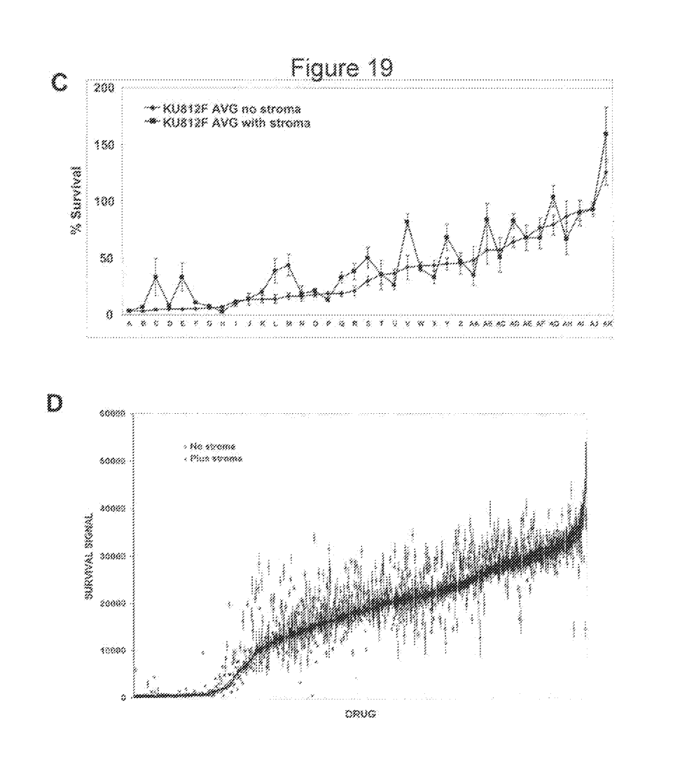

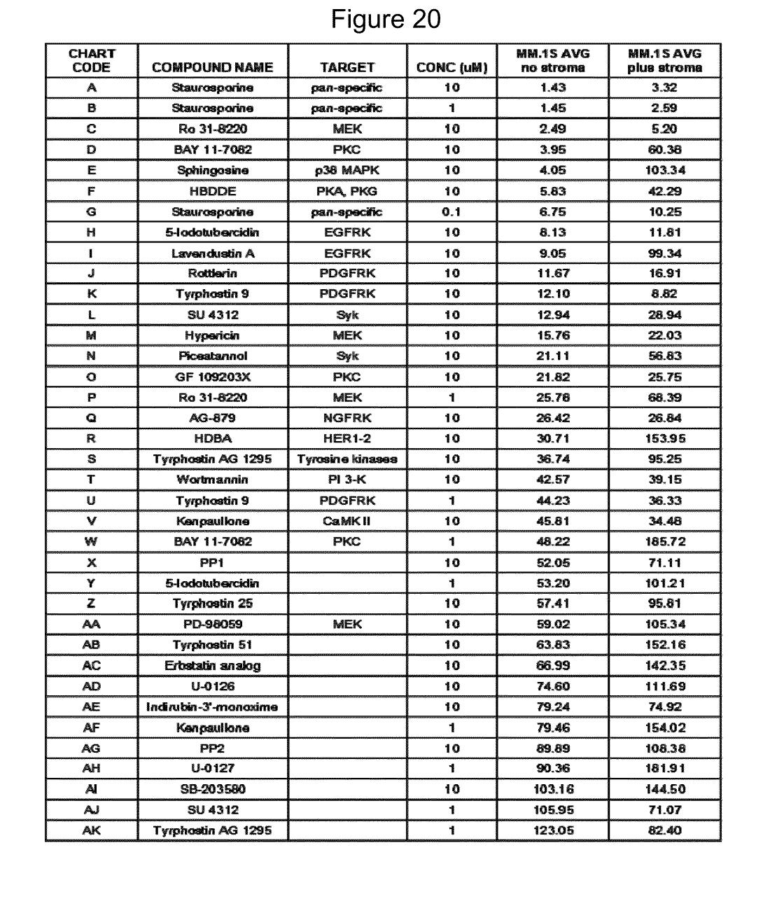

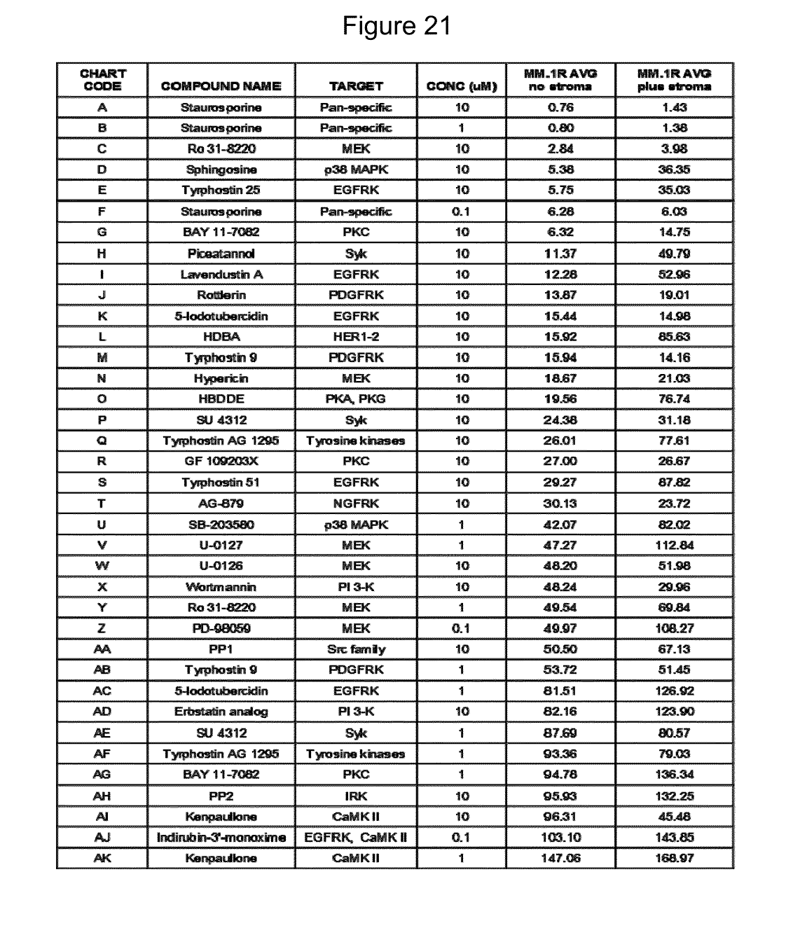

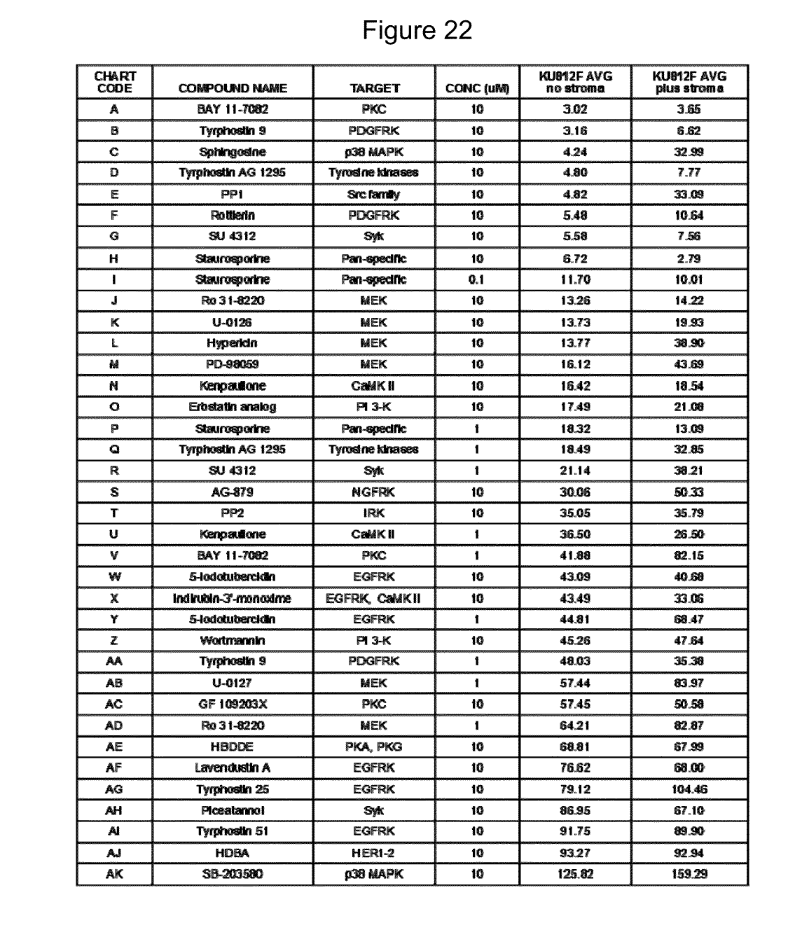

Similarly, HS-5 stroma conferred protection to MM1S-GFP-luc cells against Doxo treatment (0-250 ng/mL), but IL-6 blocking antibody (e.g., those from R&D Systems Cat. No. AF-227-NA) appeared to antagonize the protective effect (data not shown). Blocking IL-6 by using IL-6 antibody alone (without stromal cells) did not appear to have any effect against Doxo treatment (FIG. 9A). This suggested that IL-6 antibody may be an agent that can overcome the stromal cell-mediated tumor resistance. In contrast, blocking the IL-6 Receptor (IL-6R) using the anti-IL-6R blocking antibody (e.g., those from R & D Systems Cat. No. AF-206-NA) did not appear to overcome the HS-5 stroma-mediated tumor resistance at low concentrations of Doxo (e.g., less than about 65 ng/mL), but appeared to overcome the HS-5 stroma-mediated tumor resistance at high concentrations of Doxo (e.g., more than about 100 ng/mL) (FIG. 9B).

In another set of experiments, Applicants tested and observed similar protective/sensitization effect of stroma cell on KU812F-luc cells on various drugs (data not shown). Here, the leukemia cell line KU812F-luc was grown in RPMI 1640 medium (Bio Whittaker) supplemented with 100 U/ml penicillin, 100 μg/ml streptomycin and 10% fetal bovine serum (FBS; GIBCO/BRL, Gaithersburg, Md.).

Furthermore, when Applicants compared (by MTT assay, supra) the results of drug treatments (e.g., 0-40 nM bortezomib, 0-250 ng/mL of Doxo) of GFP/Luc-expressing MM cell litres vs. their parental untransfected cell lines. Applicants observed no difference in the survival pattern across the doses tested (FIGS. 6E and 6F). This indicates that stable genetic marking of rumor cells with luciferase or GFP/luciferase constructs can be performed without biasing the responsiveness of the tumor cells to diverse drugs of interest.

### Example IV

### Applications of In Vitro CS-BLI in Tumor-Stromal Co-Cultures in Other Malignancies

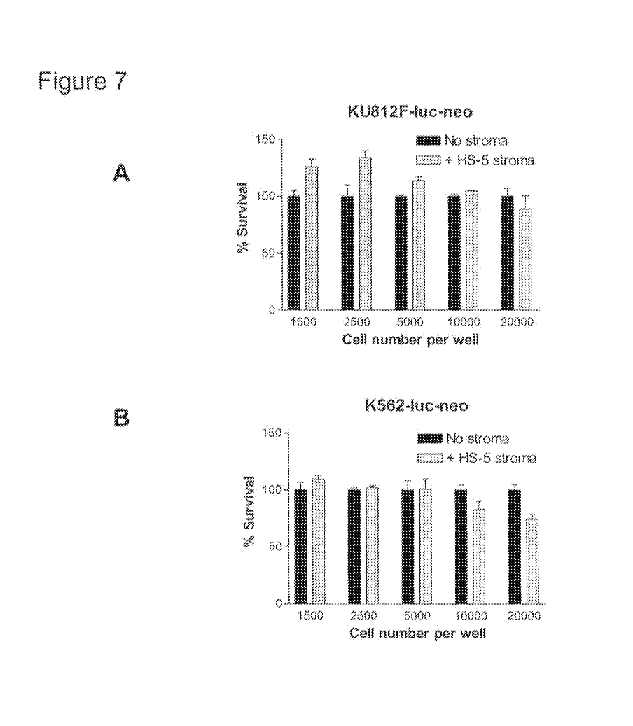

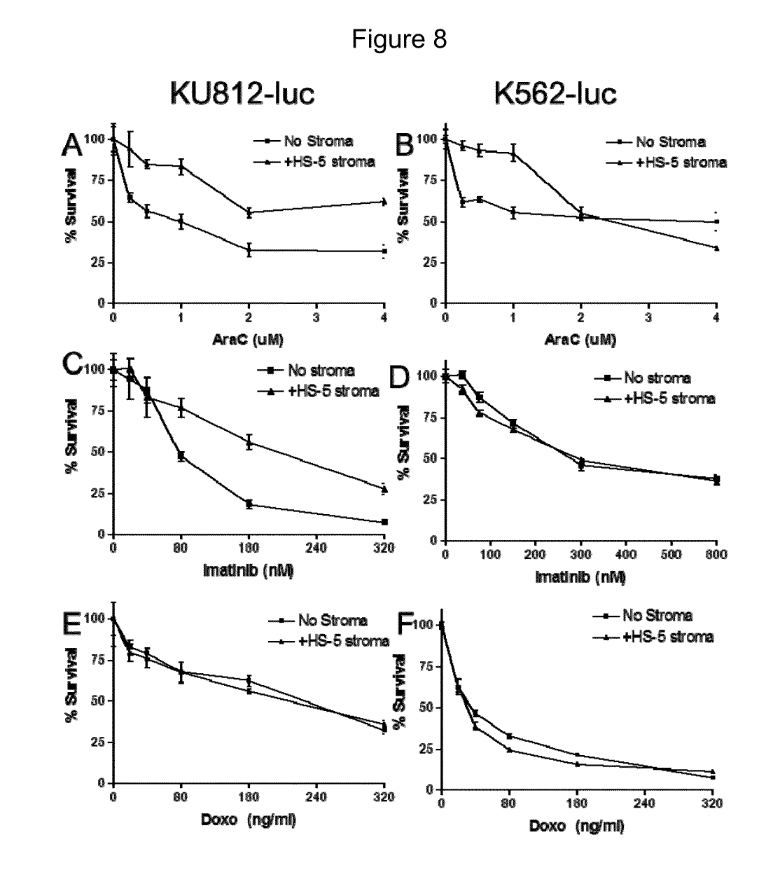

Applicants expanded the application of in vitro compartment-specific bioluminescence imaging assays beyond the MM cells to other tumor models, namely a few leukemic cell lines. Applicants specifically tested luciferase-expressing K562-luc-neo and KU812F-luc-neo leukemic cells against standard anti-leukemia agents, including AraC and Doxo, in the presence vs. absence of HS-5 stromal cells (FIGS. 7A and 7B). Here, the culture conditions and experimental settings were identical to the myeloma cells, and the leukemia cell lines K562-luc and KU812F-luc were grown in RPMI 1640 medium (BioWhittaker) supplemented with 100 U/ml penicillin, 100 μg/ml streptomycin and 10% fetal bovine serum (FBS; GIBCO/BRL, Gaithersburg, Md.). Co-culture with HS-5 cells decreased the responsiveness of KU812F-luc cells to treatment with AraC (0-4 μM) (FIG. 8A) and imatinib (0-320 nM) (FIG. 8C), but did not affect their response to Doxo (0-320 ng/mL) (FIG. 8E). When K562-luc cells were co-cultured with stromal cells, protection was observed against AraC (0-4 μM) (FIG. 8B), but not imatinib (0-320 nM) (FIG. 8D) or Doxo (0-320 ng/mL) (FIG. 8F). These results further underscore the significance of screening for new anticancer drugs in the presence of appropriate stromal support systems for tumor cells.

### Example V

### Applications of In Vitro Time-Lapse CS-BLI in Assessment of Time-Dependent Changes in Tumor Cell Response to Drug in the Presence or Absence of Stromal Cells

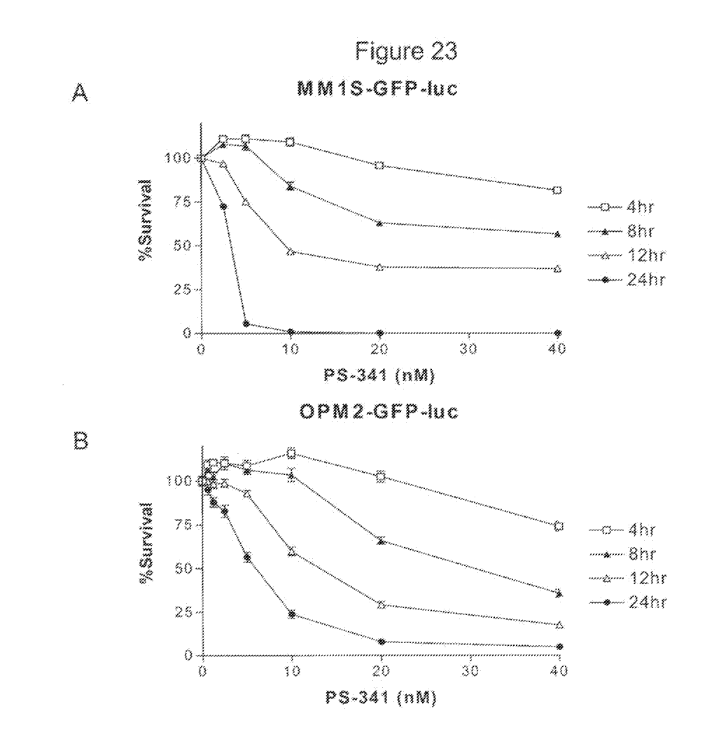

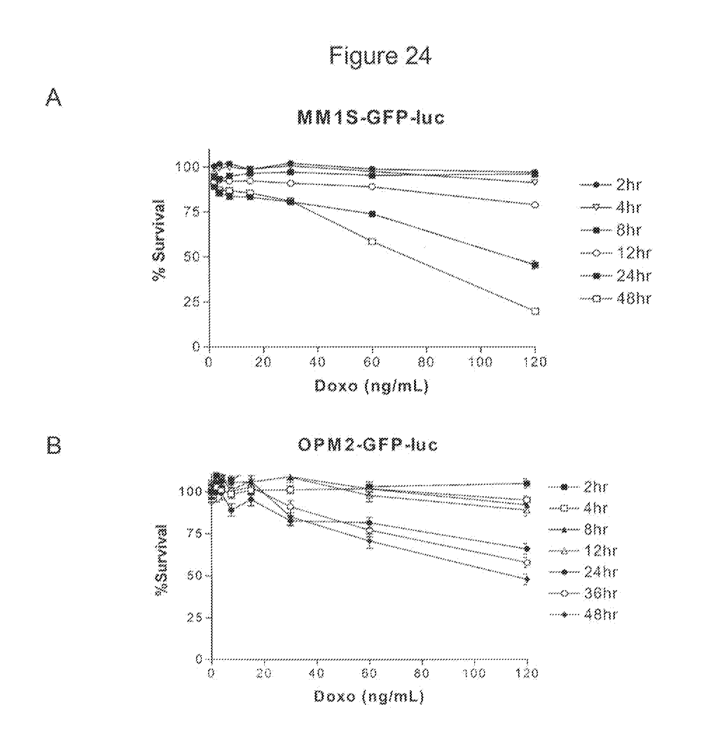

Applicants expanded the application of in vitro compartment-specific Bioluminescence imaging assays to include time-lapse measurements of tumor cell viability across various time points in the presence or absence of stromal cells. MM cell viability was measured serially in response to PS-341 across several time points in the same culture plate. The MM cell lines MM-1S-GFP-luc (FIG. 23A) and OPM-2-GFP-luc (FIG. 23B) were plated, treated with increasing doses of PS-341 and luciferin substrate added at time 0. Cell viability was assessed serially by CS-BLI up to 24 hrs after initiation of treatment and viability signal was normalized to non-drug treated controls. Similarly, MM-1S-GFP-luc (FIG. 24A) and OPM2-GFP-luc (FIG. 24B) cell viability in response to Doxorubicin was serially measured in the same plate across several time points up to 48 hrs from initiation of treatment. Compartment-specific bioluminescence signal was normalized to non-drug treated controls.

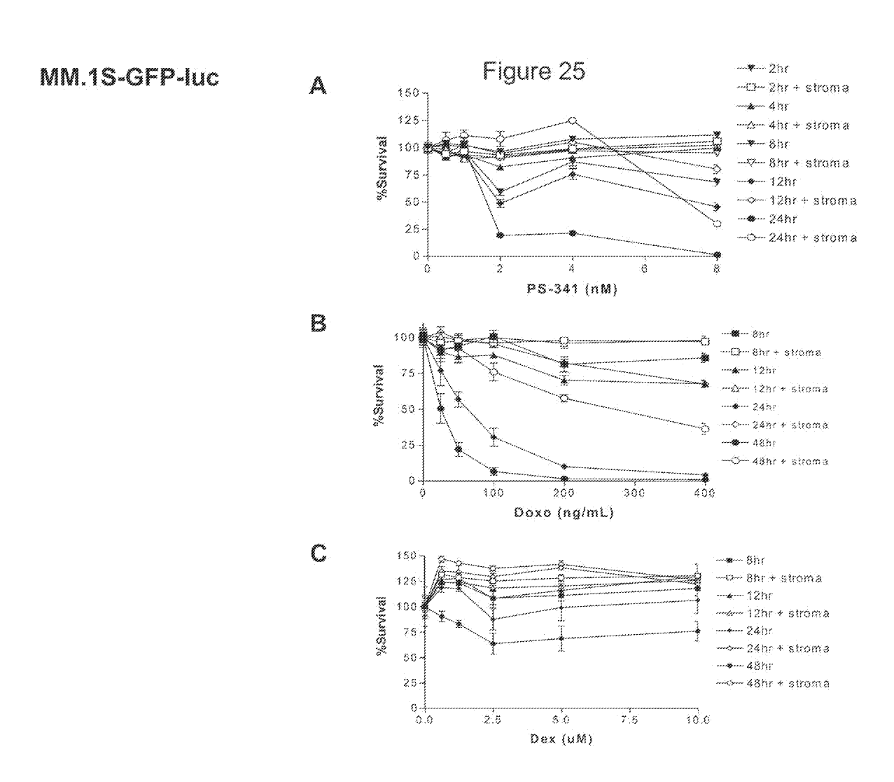

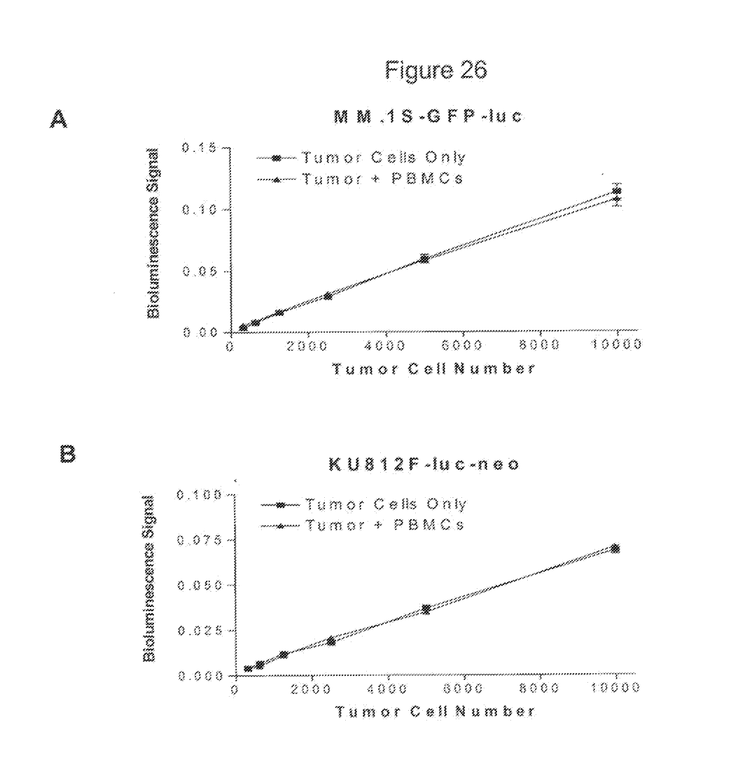

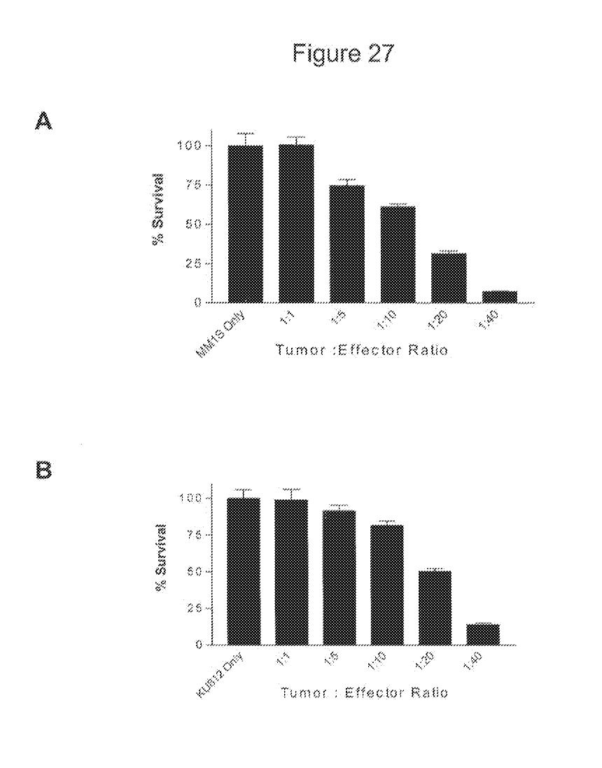

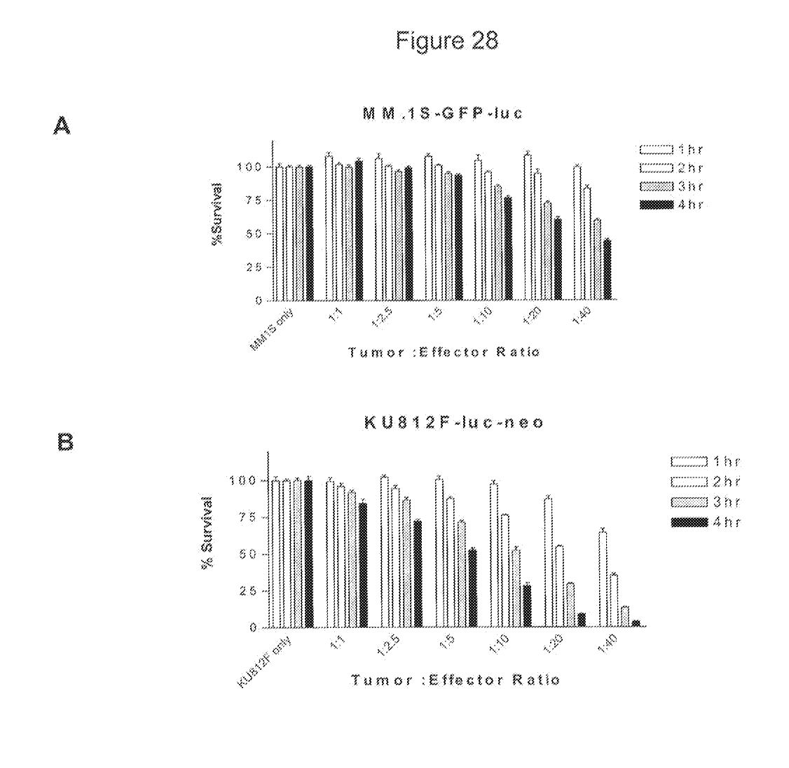

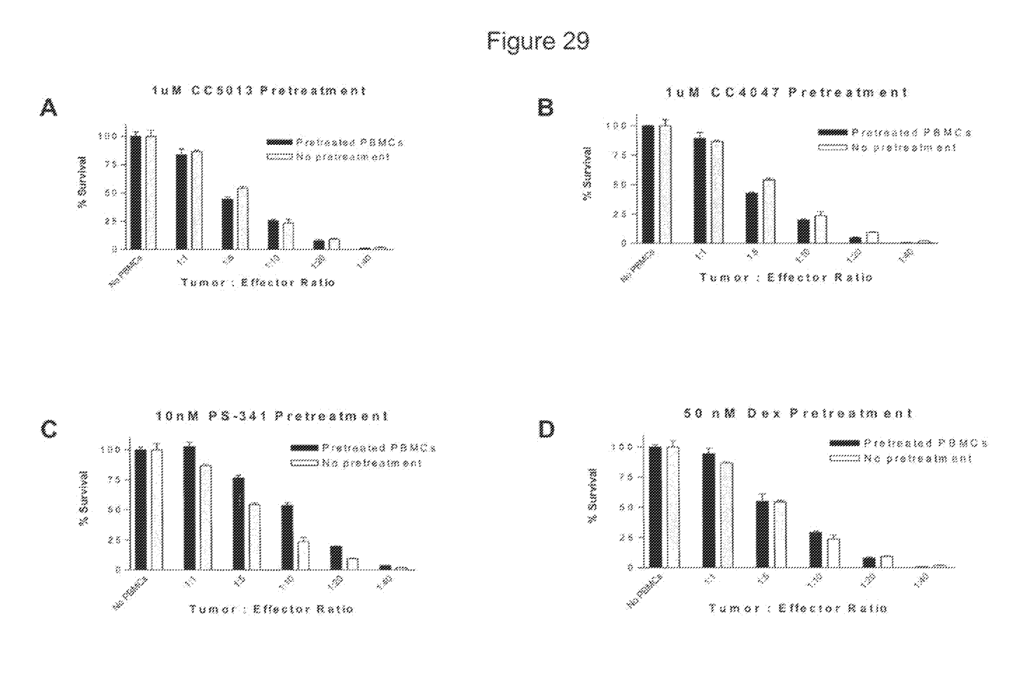

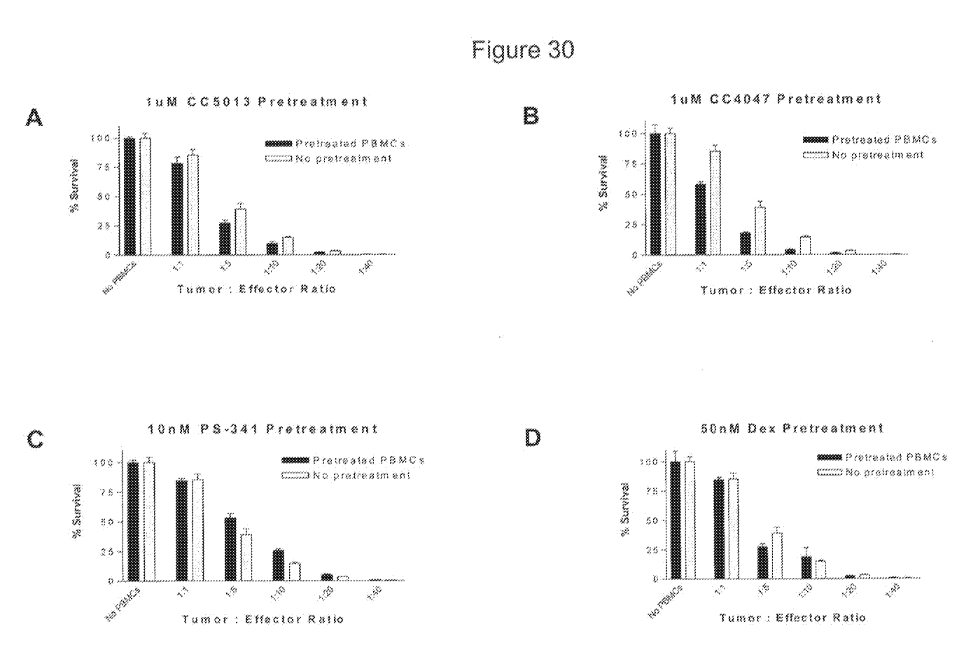

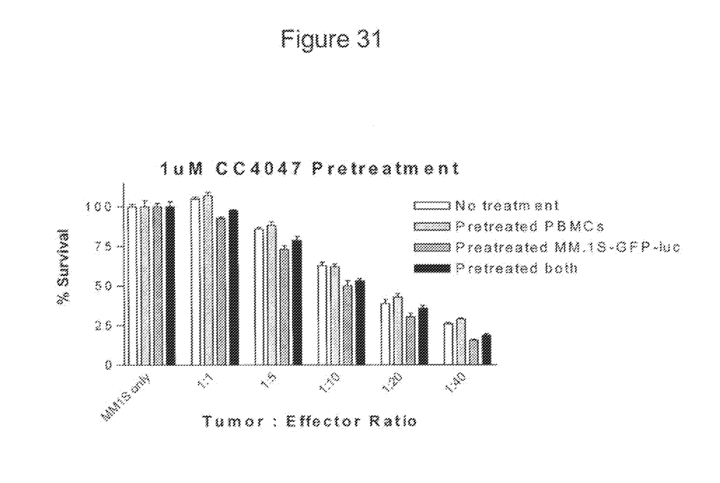

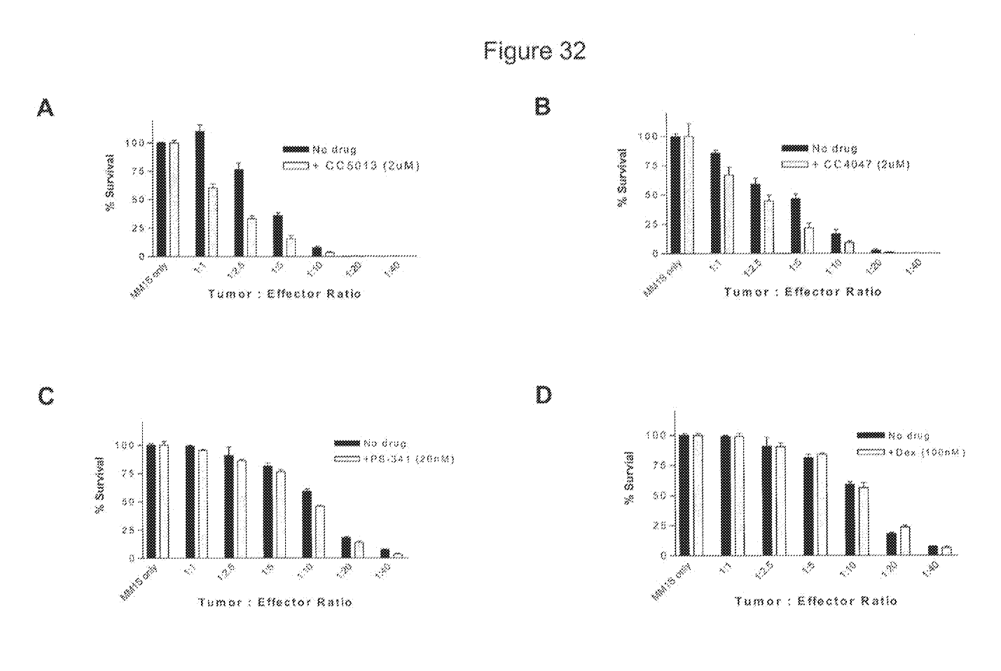

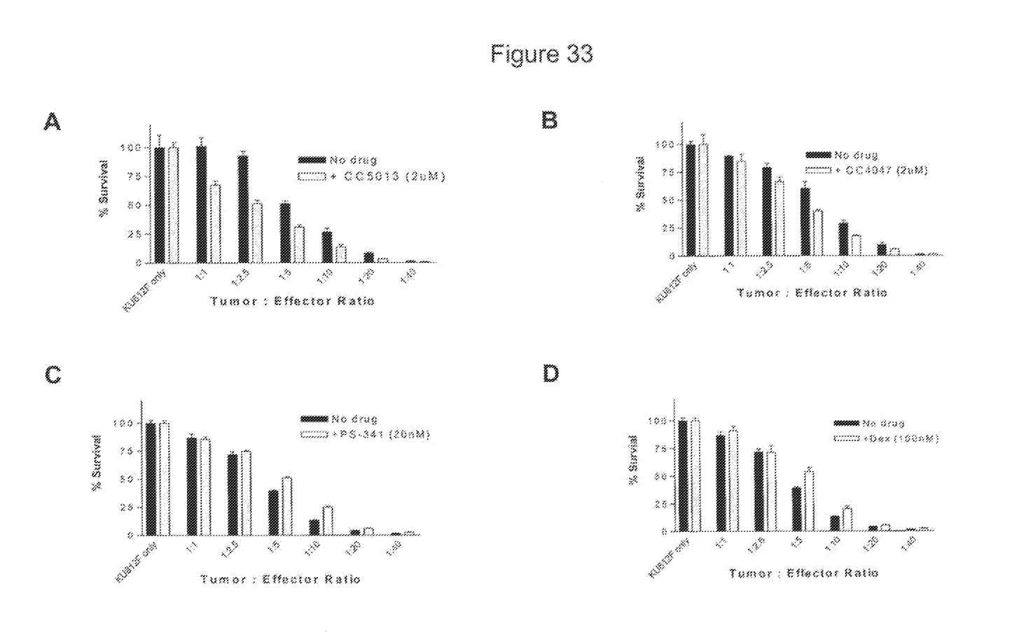

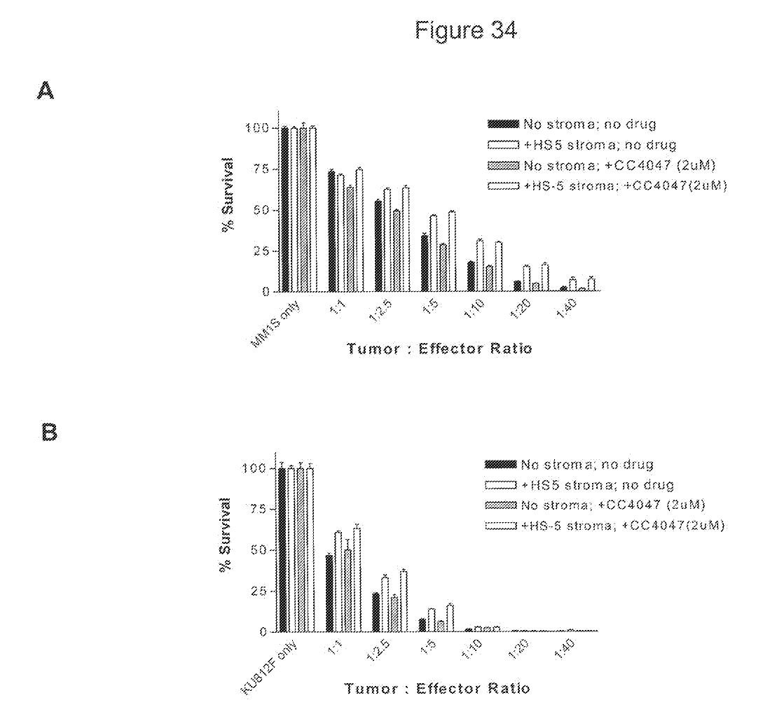

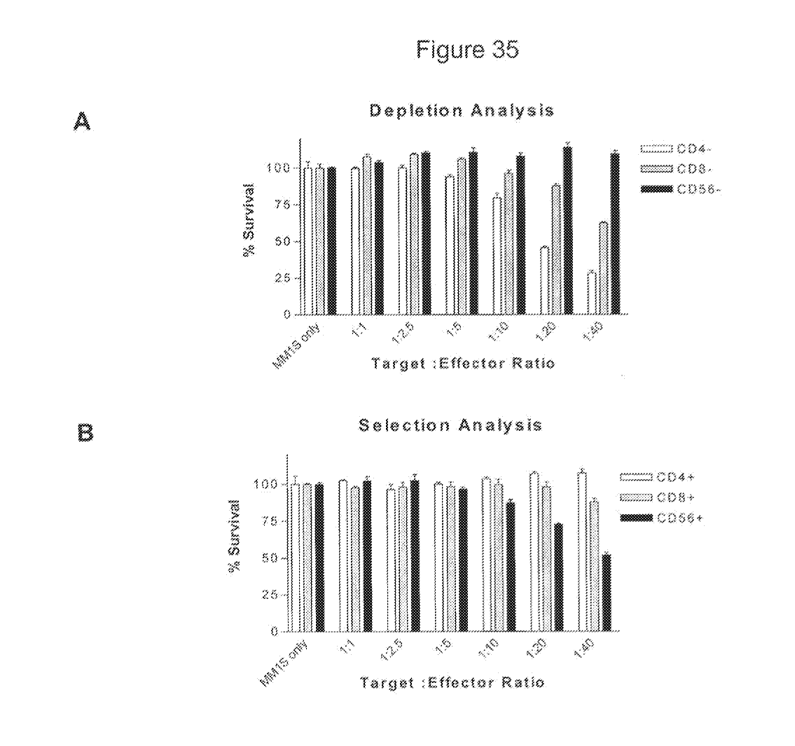

Time-lapse CS-BLI was applied for measuring MM cell viability in response to PS-341, Doxorubicin, and Dex across several time points for each culture plate in the presence vs. absence of stromal cells. Cultures were treated with increasing doses of PS-341 (FIG. 25A), Doxorubicin (FIG. 25B) or Dexamethasone (FIG. 25C), detection substrate added at time 0, and cell viability assessed serially for up to 48 hrs by measuring bioluminescence and viability signal normalized to non-drug treated controls in the absence of stromal cells. These results underscore the capability of CS-BLI, in a time-lapse fashion, to identify the detailed kinetics of anti-tumor drug responses in the presence or absence of stromal cells.

### REFERENCES

1. Mueller M M, Fusenig N E, Friends or foes—bipolar effects of the tumour stroma in cancer. Nat Rev Cancer 4: 839-849, 2004.

2. Roodman G D. Role of the bone marrow microenvironment in multiple myeloma. J Bone Miner Res. 17: 1921-1925, 2002.

3. Tost P, Tura S. Antiangiogenic therapy in multiple myeloma. Acta Haematol 106: 208-213, 2001.

4. Vincent T, Mechti N. Extracellular matrix in bone marrow can mediate drug resistance in myeloma. Leak Lymphoma, 46: 803-811, 2005.

5. Pagnuoeo G, Cardinale G, Gervasi F. Targeting multiple myeloma cells and their bone marrow microenvironment. Ann N Y Acad Sci. 1028: 390-399, 2004.

6. Hideshima T, Chauhan D, Podar K, Schlossman R L, Richardson P, Anderson K C. Novel therapies targeting the myeloma cell and its bone marrow microenvironment. Semin Oncol 28: 607-612, 2001.

7. Grigorieva I, Thomas X, Epstein J. The bone marrow stromal environment is a major factor in myeloma cell resistance to dexamethasone. Exp Hematol. 26: 597-603, 1998.

8. Hurt E M, Wiestner A, Rosenwald A, et al. Overexpression of c-maf is a frequent oncogenic event in multiple myeloma that promotes proliferation and pathological interactions with bone marrow stroma. Cancer Cell. 5: 191-199, 2004.

9. Richardson P G, Schlossman R L, Weller E, et al. Immunomodulatory drug CC-5013 overcomes drug resistance and is well tolerated in patients with relapsed multiple myeloma. Blood 100: 3063-3067, 2002.

10. Kumar S, Raje N, Hideshima T, et al, Antimyleoma activity of two novel N-substituted and tetraflourinated thalidomide analogs. Leukemia 19: 1253-1261, 2005.

11. Hideshima T, Chauhan D, Shima Y, et al. Thalidomide and its analogs overcome drug resistance of human multiple myeloma cells to conventional therapy. Blood 96: 2943-2950, 2000.

The contents of all cited references (including literature references, issued patents, published patent applications as cited throughout this application) are hereby expressly incorporated by reference.

### Equivalents

Those skilled in the art will recognize, or be able to ascertain, using no more than routine experimentation, numerous equivalents to the specific method and reagents described herein, including alternatives, variants, additions, deletions, modifications and substitutions. Such equivalents are considered to be within the scope of this invention and are covered by the paragraphs in the Summary of the Invention.

1-43. (canceled)

44. A method for identifying a compound useful for modulating a cellular biological activity of interest in cells of the first cellular compartment, the method comprising:
(1) contacting the cell co-culture system of claim 1 with a test compound;
(2) detecting the signal generated by the compartment-specific marker from the cell co-culture system in the presence and absence of the test compound;
wherein a statistically significant difference in the signal after contact with the test compound compared to the signal in the absence of the test compound is indicative that the test compound is capable of modulating the cellular biological activity of interest in cells of the first cellular compartment in the presence of cells in the second compartment.

45. A method for identifying a compound useful for modulating a cellular biological activity of interest in the first cellular compartment, the method comprising:
(1) contacting the cell co-culture system of claim 1 with a test compound;
(2) contacting, under substantially the same conditions, a second cell culture comprising the first cellular compartment but not the second cellular compartment with the test compound;
(3) detecting the signal generated by the compartment-specific marker from the cell co-culture system and the second cell culture;
wherein a statistically significant decrease in the signal from the cell co-culture system compared to that of the second cell culture is indicative that the test compound is capable of modulating, the cellular biological activity of interest in cells of the first cellular compartment.

46. The method of claim 44 or 45, wherein the test compound is a synthetic compound, a natural compound, or a mixture of multiple compounds from either class thereof.

47. The method of claim 44 or 45, wherein the test compound is tested at two or more different concentrations.

48. The method of claim 44 or 45, wherein the test compound is from a chemical library, a polypeptide library, an antibody library, a small molecule library, a polynucleotide library, or a mixture of multiple compounds from any class thereof.

49. The method of claim 44 or 45, wherein the signal is a fluorescent signal.

50. The method of claim 44 or 45, wherein said cellular biological activity of interest is cell viability, cell proliferation, cell migration, cell adhesion, temporal and/or spatial organization of cell morphology, or cell differentiation.

51. The method of claim 44 or 45, further comprising determining the ability of the identified test compound to affect the activity of the compartment-specific marker, wherein an identified test compound not substantially modulating the activity of the compartment-specific marker is useful for affecting the cellular biological activity of interest.

52. The method of claim 44, wherein the step of detecting the signal generated by the compartment-specific marker from the cell co-culture system in the presence and absence of the test compound is performed at more than one time point.

53. The method of claim 45, wherein the step of detecting the signal generated by the compartment-specific marker from the cell co-culture system and the second cell culture is performed at more than one time point.

54. A method for identifying a treatment useful for modulating a cellular biological activity of interest, the method comprising:
(1) subjecting the cell co-culture system of claim 1 to said treatment;
(2) detecting the signal generated by the compartment-specific marker from the cell co-culture system in the presence and in the absence of the treatment;
wherein a statistically significant change in the signal after the treatment compared to that without the treatment is indicative that the treatment is useful for modulating the cellular biological activity of interest in cells of the first cellular compartment.

55. The method of claim 54, wherein said treatment is selected from the group consisting of radiation, light, heat, photodynamic therapy, cellular vaccine therapy, and cellular immune therapy.

56. The method of claim 45, wherein the cell co-culture system and the second cell culture are contacted by the test compound at substantially the same time.

57-66. (canceled)

67. A method of identifying a compound that overcomes accessory cell-mediated tumor cell resistance to an anti-tumor compound, the method comprising:
(1) contacting the cell co-culture system of claim 1 with a test compound and the anti-tumor compound,
wherein said first cellular compartment comprises a tumor cell, and said second cellular compartment comprises non-tumor accessory cells, and,
wherein the accessory cells confer accessory cell-mediated tumor cell resistance to the anti-tumor compound;
(2) detecting the signal generated by the compartment-specific marker from the cell co-culture system in the presence and absence of the test compound;
wherein a statistically significant change in the signal after contacting with the test compound compared to that before contacting the candidate compound is indicative that the candidate compound overcomes accessory cell-mediated tumor cell resistance to the anti-tumor drug.

68. The method of claim 67, further comprising verifying that the identified test compound does not substantially affect the signal generated by the compartment-specific marker in a manner disassociated from the biological endpoint that the marker is intended to measure from the cell co-culture system.

69-71. (canceled)

72. A method for identifying a compound useful for treating cancer, the method comprising:
(1) contacting the mammalian cell co-culture system of claim 69 with one or more candidate compounds;
(2) detecting the signal generated by the compartment-specific bioluminescent marker from the cell co-culture system in the presence and absence of the candidate compounds;
wherein a statistically significant decrease in the signal after contacting with the candidate compound compared to that before contacting with the candidate compound is indicative that the candidate compound is useful for treating cancer.

73. A method for identifying a compound useful for treating cancer, the method comprising:
(1) providing a cell co-culture system of claim 69, and in parallel, a second cell culture comprising the tumor cell compartment but not the accessory cell compartment;
(2) contacting, under substantially the same conditions, the cell co-culture system and the second cell culture with a candidate compound;
(3) detecting the signal generated by the compartment-specific bioluminescent marker from the cell co-culture system and the second cell culture;
wherein a statistically significant decrease in the signal from the cell co-culture system compared to that of the second cell culture is indicative that the candidate compound is useful for treating cancer.

74. The method of claim 72 or 73, wherein the bioluminescent marker is a luciferase marker.

75. The method of claim 72 or 73, wherein the bioluminescent marker is a luciferase-GFP marker.

76. The method of claim 72 or 73, wherein the bioluminescent marker is a luciferase-neo marker.

77. The method of claim 72 or 73, wherein the signal generated by the compartment-specific bioluminescent marker is detected by a bioluminescence-detecting device, a luminometer or a fluorometer.
